(19) United States
(12) Patent Application Publication
(10) Pub. No.: US 2015/0226743 A1
(43) Pub. Date: Aug. 13, 2015

(54) MULTIPLEXED METHOD FOR DIAGNOSING CLASSICAL HODGKIN LYMPHOMA

(71) Applicant: CLARIENT DIAGNOSTIC SERVICES, INC., ALISO VIEJO, CA (US)

(72) Inventors: Lawrence M. Weiss, Aliso Viejo, CA (US); Rodney A. Beck, Huntsville, AL (US); Lakshmi Sireesha Kaanumalle, Niskayuna, NY (US); Antti E. Seppo, Niskayuna, NY (US); David Lavan Henderson, Niskayuna, NY (US); Alex David Corwin, Niskayuna, NY (US); Kevin Bernard Kenny, Niskayuna, NY (US); Colin Craig Mcculloch, Niskayuna, NY (US); Fiona Ginty, Niskayuna, NY (US); Alexanderr G. Bordwell, Huntsville, AL (US); Michael S. Lazare, Niskayuna, NY (US); Denise A. Hollman-Hewgley, Aliso Viejo, CA (US)

(73) Assignee: CLARIENT DIAGNOSTICS SERVICES, INC., ALISO VIEJO, CA (US)

(21) Appl. No.: 14/402,816

(22) PCT Filed: May 30, 2013
(86) PCT No.: PCT/US2013/043409

§ 371 Date: Nov. 21, 2014

Related U.S. Application Data
(60) Provisional application No. 61/719,428, filed on Oct. 28, 2012.
(60) Provisional application No. 61/733,990, filed on Dec. 6, 2012.

(51) Int. Cl. G01N 33/574

(52) U.S. Cl. CPC G01N 33/57407; G01N 2333/70578; G01N 2333/70596; G01N 2333/70589; G01N 2333/7051; G01N 2333/47; G01N 2458/00

(57) ABSTRACT

A method of analyzing a biological sample suspected of having classic Hodgkin lymphoma, comprising (1) detecting, in a single sample, for the expression of at least two biomarkers selected from CD30, CD15, CD20, CD45, CD3, Pax-5, CD79A, BOB1 and OCT-2; and (b) analyzing the sample based on the presence, absence and/or expression level of the at least two biomarkers. Also provided is a method wherein all the nine biomarkers are analyzed on a single sample. Further provided are method for diagnosing classical Hodgkin lymphoma, as well as system and kit that comprise the means for executing the novel methods.

### FIELD OF THE INVENTION

The present invention is directed to the detection of biomarkers on a biological sample. More specifically, the present invention is directed to the diagnosis of classical Hodgkin lymphoma using a multiplexed assay method which enables the fluorescent detection of multiple biomarkers on the same section of a biological sample. Also provided are a kit and a system for performing the novel method.

### BACKGROUND OF THE INVENTION

Classical Hodgkin lymphoma, also known as Hodgkin's lymphoma and previously known as Hodgkin's disease, is a type of lymphoma, which is a cancer originating from lymphocytes. Hodgkin lymphoma is characterized by the orderly spread of disease from one lymph node group to another and by the development of systemic symptoms with advanced disease. When Hodgkin cells are examined microscopically, the presence of multinucleate Reed-Sternberg cells (RS cells, or RSC) and mononuclear variants, Hodgkin cells (H cells), is the characteristic histopathologic finding. Hodgkin lymphoma may be treated with radiation therapy, chemotherapy, or hematopoietic stem cell transplantation, with the choice of treatment depending on the age and sex of the patient and the stage, bulk, and histological subtype of the disease.

Classical Hodgkin lymphoma must be distinguished from non-cancerous causes of lymph node swelling (such as various infections) and from other types of cancer, particularly non-Hodgkin lymphoma. Definitive diagnosis is often established by lymph node biopsy (usually excisional biopsy with microscopic examination). Blood tests are also performed to assess function of major organs and to assess safety for chemotherapy. Positron emission tomography (PET) is used to detect small deposits that do not show on CT scanning (e.g., the chest, abdomen and pelvis). PET scans are also useful in functional imaging (by using a radiolabeled glucose to image tissues of high metabolism).

Microscopic examination of the lymph node biopsy reveals complete or partial effacement of the lymph node architecture by scattered large malignant cells (RS/H cells) admixed within a reactive cell infiltrate composed of variable proportions of lymphocytes, histiocytes, eosinophils, and plasma cells. The malignant cells only account for ˜1 in every hundred cells in most lymph node biopsy specimens.

Characteristics of classical Reed-Sternberg cells include large size (20-50 micrometres), abundant, amphophilic, finely granular/homogeneous cytoplasm; two mirror-image nuclei (owl eyes) each with an eosinophilic nucleolus and a thick nuclear membrane (chromatin is distributed close to the nuclear membrane). The Hodgkin cell is a variant of RS cell, which has the same characteristics, but is mononucleated.

The diagnosis of classical Hodgkin lymphoma may be very difficult because cells resembling RS/H cells may be associated with other diseases, e.g., reactive lymphadenopathy or non-Hodgkin lymphomas. (Weiss L M, Warnke R A, Hansmann M-L, Chan J K C, Muller-Hermelink H K, Harris N, Jaffe E: Pathology of Hodgkin lymphoma. In: Hodgkin Lymphoma, 2^{nd} Edition. Ed. By Hoppe R, March P, Armitage J, Diehl V, Weiss L M. Lippincott Williams & Wilkins, Philadelphia, 2007, pp. 43-71.)

For many patients, an informative diagnosis requires the observation of multiple stains to assess the characteristics of the RS/H cells. The RS/H cells have a characteristic pattern of reactivity with the antibodies against CD30, CD15, CD45, CD20 and others. This is difficult to assess in serial slides and could consume a dozen slides and hamper a diagnosis of Hodgkin lymphoma. Some of the key challenges of this technique are that serial immunostains are used and hence it can be difficult or impossible to locate the same Hodgkin cell on adjacent slides. Moreover, the expression profile of the Hodgkin cells in an individual case is often variable, requiring the performance of a large battery of markers. As a consequence, about a third of classical Hodgkin lymphoma cases are considered difficult to diagnose. Better differentiation of classical Hodgkin lymphoma from other conditions (e.g. benign lymph node inflammation, B-cell lymphoma, T-cell lymphoma) is needed.

Given the rarity of the Hodgkin cells coupled with the number of markers that are needed for a definitive diagnosis, the inventors have recognized a need to develop a new, multiplexed assay technique in which a single patient slide is multiplexed with multiple different antibodies. The novel method allows comprehensive assessment of the staining characteristics of individual well-preserved RS/H cells, resulting in fewer inconclusive diagnoses. It also allows for easy visualization of multiple markers on this unique cell type.

### SUMMARY OF THE INVENTION

The invention includes embodiments that provide innovative classical Hodgkin lymphoma diagnostics which enable qualitative and quantitative assessment of biomarkers and allow for visualization of biomarker co-localization at the cellular level on a single slide. This provides greater precision and true concordance between biomarker signals, thus enhancing the information provided by pathologists to oncologists, resulting in better patient care. The method is suited for the diagnosis of classical Hodgkin lymphoma, in which often only a small amount of tissue is available for testing. Similarly, the method could be adapted to suit the diagnosis of other diseases where only a small or rare tissue sample is obtainable or only rare atypical cells are present in the biopsy.

In one aspect of the invention, it is provided a method for generating a composite image of a single tissue sample from a classical Hodgkin lymphoma patient. Thus, in one embodiment, it is provided a method for providing a composite image which comprises: (1) generating a first series of images of the biological sample, which step comprises: (a) contacting the sample on a solid support with a first binder for a first target biomarker; (b) staining the sample with a fluorescent marker that provides morphological information; (c) detecting, by fluorescence, signals from the first binder and the fluorescent marker; and (d) generating the first images of at least part of the sample from the detected fluorescent signals; (2) after signal removal from the first binder, generating one or more second series of images of the biological sample, which step comprises; (a) contacting the same sample with a binder for another target biomarker; (b) optionally staining the sample with a fluorescent marker that provides morphological information; (c) detecting, by fluorescence, signals from the binder and the fluorescent marker; and (d) generating the second images of at least part of the sample from the detected fluorescent signals; and (3) generating a composite image that provides the relative location or expression of both the first target biomarkerand the other biomarker.

In another aspect of the invention, it is provided a method for analyzing a biological sample, which method comprises providing a composite image of the biological sample according to certain aspects of the invention, and analyzing the presence and expression level of the biomarkers of interest from the composite image. In certain embodiments, the method further comprises creating a RGB color blend heatmap image of the biomarker expression level by mapping the fluorescent signal from each of the binders for each of the biomarkers to a reference color lookup table. In certain other embodiments, the method further comprises creating color blended composite images for each of the images, the images include the image of the biomarker expression and the fluorescent marker distribution.

In another aspect of the invention, it is provided a method of diagnosing a classical Hodgkin lymphoma, which method comprises analyzing a biological sample according to certain aspects of the invention, and diagnosing whether the patient has a classical Hodgkin lymphoma.

In another aspect of the invention, it is provided a method of analyzing a biological sample suspected of having classic Hodgkin lymphoma, comprising: detecting, in a single sample, for the expression of at least two biomarkers selected from CD30, CD15, CD20, CD45, CD3, Pax-5, CD79A, BOB1 and OCT-2; and analyzing the sample based on the presence, absence and/or expression level of the at least two biomarkers.

In another aspect of the invention, it is provided a modified tissue sample suspected of having classic Hodgkin lymphoma, the modification includes that the sample was contacted by a binder for each of at least two biomarkers selected from CD30, CD15, CD20, CD45, CD3, Pax-5, CD79A, BOB1 and OCT-2; wherein the binders are optionally detectable by fluorescence. In certain embodiments, the modified tissue sample was contacted by a binder for each of at least three of the biomarkers. In certain other embodiments, the modified tissue sample was contacted by a binder for each of at least four of the biomarkers. In certain embodiments, the modified tissue sample was contacted by a binder for each of at least CD30, CD15, CD45, CD3 and Pax-5. In still other embodiments, the modified tissue sample was contacted by a binder for at least each of the nine biomarkers CD30, CD15, CD20, CD45, CD3, Pax-5, CD79A, BOB1 and OCT-2. In certain embodiments, the binders are specific antibodies for each of the biomarkers.

In still another aspect of the invention, it is provided a kit for the fluorescent detection of at least two biomarkers on the same biological sample. Thus, an embodiment of the invention provides a kit that includes components for performing the novel method of the invention.

In yet another aspect of the invention, it is provided a system for the fluorescent detection of at least two biomarkers on the same biological sample. Thus, one embodiment of the invention provides a system that includes means for performing the novel method of the invention.

In another aspect of the invention, it is provided a kit for the diagnosis of classical Hodgkin lymphoma. Thus, one embodiment of the invention provides a kit comprising a diagnostic panel of antibodies that includes: a first antibody that binds to CD30; and a second antibody that binds to a biomarker selected from the group consisting of CD45, CD 15, CD3, CD20, Pax-5, CD79A, BOB1 and OCT-2. In another embodiment, it is provided a kit comprising a diagnostic panel of antibodies that comprises antibodies that bind to each of CD30, CD45, CD15, CD3, Pax-5. In yet another embodiment, it is provided a kit comprising a diagnostic panel of antibodies that includes antibodies that bind to each of CD30, CD45, CD15, CD3, CD20, Pax-5, CD79A, BOB1 and OCT-2.

In still another aspect of the invention, it is provided a method of treatment for a patient having classical Hodgkin lymphoma, comprising diagnosing the patient as having classical Hodgkin lymphoma according to certain embodiments of the invention, and treating the patient with a drug for classical Hodgkin lymphoma. In certain embodiments, the drug targets CD30. In certain other embodiments, the drug is selected from apomab (RG7425) by Roche, brentuximab vedotin (SGN-35) by Seattle Genetics & Takeda, DCDT2980S Roche & Seattle Genetics, PF-05230905 Pfizer & Ablynx and tigatuzumab (CS-1008) Daiichi Sankyo.

### BRIEF DESCRIPTION OF THE DRAWINGS

FIG. 1 is a flowchart showing the key steps for an embodiment of the invention. Steps in brackets are optional.

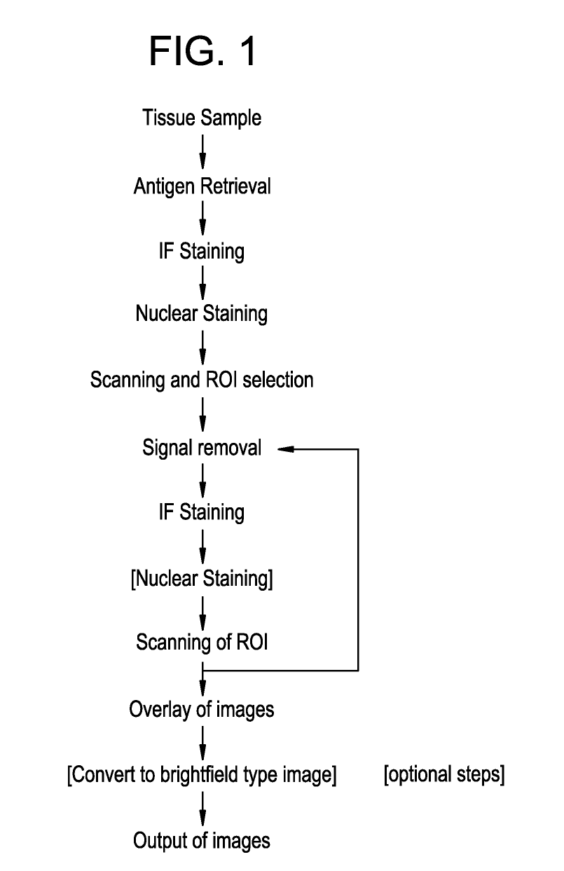

FIG. 2 shows representative 10× images from a Hodgkin lymphoma sample illustrating the various visualization techniques presented to the pathologist. All images presented are from the same field of view in the tissue sample. A: CD 30 staining presented as a virtual DAB image. The brown (darker) staining is the CD30 staining and the blue (lighter) color represent the nuclei (psuedo hematoxylin staining from DAPI staining). B: CD 30 staining presented as a monochromatic, grayscale image. The gray color represents the CD30 positive areas. C. A virtual H&E image which shows the overall morphology of the tissue.

FIG. 3 shows representative 40× images from a Hodgkin lymphoma sample comparing the two fundamental ways that a single biomarker can be presented to a pathologist. Both images presented are from the same field of view in the tissue sample. A: CD 30 staining presented as a virtual DAB image. The brown (darker) staining is the CD30 staining and the blue (lighter) color represent the nuclei (psuedo hematoxylin staining from DAPI staining). B: CD 30 staining presented as a monochromatic, grayscale image. The gray color represents the CD30 positive areas.

FIG. 4 shows representative 40× virtual DAB images from a Hodgkin lymphoma sample illustrating the results of multiplexing all nine antibodies on a single tissue section. The nine antibodies evaluated were: CD30, CD15, CD45, Pax5, CD20, CD79a, Oct2, BOB1, and CD3. The brown (darker) color represents areas that are positive for that particular biomarker and the blue (lighter) color represents the nuclei (pseudo hematoxylin from DAPI staining) staining. Images presented are all from the same field of view in the tissue sample

FIG. 5 shows representative 40× images from a Hodgkin lymphoma. Row1: CD30 and CD15 shown as a monochromatic grayscale image then as a blended overlay of both channels. In the blended overlay, CD30 is represented in yellow and CD15 is green. Row2: CD30 and Pax5 shown as a monochromatic grayscale image then as a blended overlay of both channels. In the blended overlay, CD30 is represented in yellow and Pax5 in purple. Row3: CD30 and CD45 shown as a monochromatic grayscale image then as a blended overlay of both channels. In the blended overlay, CD30 is represented in yellow and CD45 is red. All images are from the same field of view in the tissue.

### DETAILED DESCRIPTION OF THE INVENTION

To more clearly and concisely describe and point out the subject matter of the claimed invention, the following definitions are provided for specific terms that are used in the following description and the claims appended hereto.

The singular forms “a” “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Unless otherwise indicated, all numbers expressing quantities of ingredients, properties such as molecular weight, reaction conditions, so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least each numerical parameter should at least be construed in light of the number of reported significant digits and by applying ordinary rounding techniques.

As used herein, the term “solid support” refers to an article on which the biological sample may be immobilized and the biomarker (e.g., protein or nucleic acid sequence) may be subsequently detected by the methods disclosed herein. The biological sample may be immobilized on the solid support by physical adsorption, by covalent bond formation, or by combinations thereof. A solid support may include a polymeric, a glass, or a metallic material. Examples of solid supports include a membrane, a microtiter plate, a bead, a filter, a test strip, a slide, a cover slip, and a test tube.

As used herein, the term “fluorescent marker” refers to a fluorophore that selectively stains particular subcellular compartments. Examples of suitable fluorescent marker (and their target cells, subcellular compartments, or cellular components if applicable) may include, but are not limited to: 4′,6-diamidino-2-phenylindole (DAPI) (nucleic acids), Eosin (alkaline cellular components, cytoplasm), Hoechst 33258 and Hoechst 33342 (two bisbenzimides) (nucleic acids), Propidium Iodide (nucleic acids), Quinacrine (nucleic acids), Fluorescein-phalloidin (actin fibers), Chromomycin A 3 (nucleic acids), Acriflavine-Feulgen reaction (nucleic acid), Auramine O-Feulgen reaction (nucleic acids), Ethidium Bromide (nucleic acids). Nissl stains (neurons), high affinity DNA fluorophores such as POPO, BOBO, YOYO and TOTO and others, and Green Fluorescent Protein fused to DNA binding protein (e.g., histones), ACMA, and Acridine Orange. Preferably, the fluorescent marker stains the nucleus.

As used herein, the term “fluorophore” refers to a chemical compound, which when excited by exposure to a particular wavelength of light, emits light (at a different wavelength). The terms “fluorescence”, “fluorescent”, or “fluorescent signal” all refer to the emission of light by an excited fluorophore. Fluorophores may be described in terms of their emission profile, or “color.” Green fluorophores (for example Cy3, FITC, and Oregon Green) may be characterized by their emission at wavelengths generally in the range of 515-540 nanometers. Red fluorophores (for example Texas Red, Cy5, and tetramethylrhodamine) may be characterized by their emission at wavelengths generally in the range of 590-690 nanometers. Examples of fluorophores include, but are not limited to, 4-acetamido-4′-isothiocyanatostilbene-2,2′disulfonic acid, acridine, derivatives of acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin, coumarin derivatives, 7-amino-4-methylcoumarin (AMC, Coumarin 120), 7-amino-trifluoromethylcouluarin (Coumaran 151), cyanosine; 4′,6-diaminidino-2-phenylindole (DAPI), 5′,5″-dibromopyrogallol-sulfonephthalein (Bromopyrogallol Red), 7-diethylamino-3-(4′-isothiocyanatophenyl)4-methylcoumarin, -, 4,4′-diisothiocyanatodihydro-stilbene-2,2′-disulfonic acid, 4,4′-diisothiocyanatostilbene-2,2′-disulfonic acid, 5-[dimethylamino]naphthalene-1-sulfonyl chloride (DNS, dansyl chloride), eosin, derivatives of eosin such as eosin isothiocyanate, erythrosine, derivatives of erythrosine such as erythrosine B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC); fluorescamine derivative (fluorescent upon reaction with amines); IR144; IR1446; Malachite Green isothiocyanate; 4-methylumbelliferone; ortho cresolphthalein; nitrotyrosine; pararosaniline; Phenol Red, B-phycoerythrin; o-phthaldialdehyde derivative (fluorescent upon reaction with amines); pyrene and derivatives such as pyrene, pyrene butyrate and succinimidyl 1-pyrene butyrate; Reactive Red 4 (Cibacron™ Brilliant Red 3B-A), rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl Rhodamine, tetramethyl rhodamine isothiocyanate (TRITC); riboflavin; rosolic acid and lathanide chelate derivatives, quantum dots, cyanines, and squaraines.

In some embodiments, a fluorophore may essentially include a fluorophore that may be attached to an antibody, for example, in an immunofluorescence analysis. Suitable fluorophores that may be conjugated to an antibody include, but are not limited to, Fluorescein, Rhodamine, Texas Red, Cy2, Cy3, Cy5, VECTOR Red, ELF™ (Enzyme-Labeled Fluorescence), Cy2, Cy3, Cy3.5, Cy5, Cy7, FluorX, Calcein, Calcein-AM, CRYPTOFLUOR™'S, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-[6-(7-nitrobenz-2-oxa-1,3-diazol-4-yl)amino]caproyl](NBD), BODIPY, boron dipyrromethene difluoride, Oregon Green, MITOTRACKER, Red, DiOC.sub.7 (3), DiIC.sub.18, Phycoerythrin, Phycobiliproteins BPE (240 kDa) RPE (240 kDa) CPC (264 kDa) APC (104 kDa), Spectrum Blue, Spectrum Aqua, Spectrum Green, Spectrum Gold, Spectrum Orange, Spectrum Red, NADH, NADPH, FAD, Infra-Red (IR) Dyes, Cyclic GDP-Ribose (cGDPR), Calcofluor White, Lissamine, Umbelliferone, Tyrosine or Tryptophan. In some embodiments, a fluorophore may essentially include a cyanine dye. In some embodiments, a fluorophore may essentially include one or more cyanine dye (e.g., Cy3 dye, a Cy5 dye, or a Cy7 dye).

“Target” or “Biomarker” as used herein, generally refers to the component of a biological sample that may be detected when present in the biological sample. The target or biomarker may be any substance for which there exists a naturally occurring specific binder (e.g., an antibody), or for which a specific binder may be prepared (e.g., a small molecule binder). In general, the binder may bind to target through one or more discrete chemical moieties of the target or a three-dimensional structural component of the target (e.g., 3D structures resulting from peptide folding). The target or biomarker may include one or more of peptides, proteins (e.g., antibodies, affibodies, or aptamers), nucleic acids (e.g., polynucleotides, DNA, RNA, or aptamers); polysaccharides (e.g., lectins or sugars), lipids, enzymes, enzyme substrates, ligands, receptors, antigens, or haptens. In some embodiments, targets may include proteins or nucleic acids.

As used herein, the term “binder” refers to a biological molecule that may bind to one or more targets in the biological sample. A binder may specifically bind to a target. Suitable binders may include one or more of natural or modified peptides, proteins (e.g., antibodies, affibodies, or aptamers), nucleic acids (e.g., polynucleotides, DNA, RNA, or aptamers); polysaccharides (e.g., lectins, sugars), lipids, enzymes, enzyme substrates or inhibitors, ligands, receptors, antigens, haptens, and the like. A suitable binder may be selected depending on the sample to be analyzed and the targets available for detection. For example, a target in the sample may include a ligand and the binder may include a receptor or a target may include a receptor and the probe may include a ligand. Similarly, a target may include an antigen and the binder may include an antibody or antibody fragment or vice versa. In some embodiments, a target may include a nucleic acid and the binder may include a complementary nucleic acid. In some embodiments, both the target and the binder may include proteins capable of binding to each other. In some embodiments, the target biomarker may be a polysaccharide and the binder may include an antibody capable of binding to the polysaccharides.

As used herein, the term “antibody” refers to an immunoglobulin that specifically binds to and is thereby defined as complementary with a particular spatial and polar organization of another molecule. The antibody may be monoclonal or polyclonal and may be prepared by techniques that are well known in the art such as immunization of a host and collection of sera (polyclonal), or by preparing continuous hybrid cell lines and collecting the secreted protein (monoclonal), or by cloning and expressing nucleotide sequences or mutagenized versions thereof, coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b and IgG3, IgM. Functional antibody fragments may include portions of an antibody capable of retaining binding at similar affinity to full-length antibody (for example, Fab, Fv and F(ab′)2, or Fab′). In addition, aggregates, polymers, and conjugates of immunoglobulins or their fragments may be used where appropriate so long as binding affinity for a particular molecule is substantially maintained.

As used herein, the term “specific binding” refers to the specific recognition of one of two different molecules for the other compared to substantially less recognition of other molecules. The molecules may have areas on their surfaces or in cavities giving rise to specific recognition between the two molecules arising from one or more of electrostatic interactions, hydrogen bonding, or hydrophobic interactions. Specific binding examples include, but are not limited to, antibody-antigen interactions, enzyme-substrate interactions, polynucleotide interactions, and the like. In some embodiments, a binder molecule may have an intrinsic equilibrium association constant (KA) for the target no lower than about 10⁵ M⁻¹ under ambient conditions (i.e., a pH of about 6 to about 8 and temperature ranging from about 0° C. to about 37° C.).

As used herein, the term “in situ” generally refers to an event occurring in the original location, for example, in intact organ or tissue or in a representative segment of an organ or tissue. In some embodiments, in situ analysis of targets may be performed on cells derived from a variety of sources, including an organism, an organ, tissue sample, or a cell culture. In situ analysis provides contextual information that may be lost when the target is removed from its site of origin. Accordingly, in situ analysis of targets describes analysis of target-bound probe located within a whole cell or a tissue sample, whether the cell membrane is fully intact or partially intact where target-bound probe remains within the cell. Furthermore, the methods disclosed herein may be employed to analyze targets in situ in cell or tissue samples that are fixed or unfixed.

A “chemical agent” may include one or more chemicals capable of modifying the fluorophore or the cleavable linker (if present) between the fluorophore and the binder. A chemical agent may be contacted with the fluorophore in the form of a solid, a solution, a gel, or a suspension. Suitable chemical agents useful to modify the signal include agents that modify pH (for example, acids or bases), electron donors (e.g., nucleophiles), electron acceptors (e.g., electrophiles), oxidizing agents, reducing agents, or combinations thereof. In some embodiments, a chemical agent may include a base, for example, sodium hydroxide, ammonium hydroxide, potassium carbonate, or sodium acetate. In some embodiments, a chemical agent may include an acid, for example, hydrochloric acid, sulfuric acid, acetic acid, formic acid, trifluoroacetic acid, or dichloroacetic acid. In some embodiments, a chemical agent may include nucleophiles, for example, cyanides, phosphines, or thiols. In some embodiments, a chemical agent may include reducing agents, for example, phosphines, thiols, sodium dithionite, or hydrides that can be used in the presence of water such as borohydride or cyanoborohydrides. In some embodiments, a chemical agent may include oxidizing agents, for example, active oxygen species, hydroxyl radicals, singlet oxygen, hydrogen peroxide, or ozone. In some embodiments, a chemical agent may include a fluoride, for example tetrabutylammonium fluoride, pyridine-HF, or SiF₄.

One or more of the aforementioned chemical agents may be used in the methods disclosed herein depending upon the susceptibility of the fluorophore, of the binder, of the target, or of the biological sample to the chemical agent. In some embodiments, a chemical agent that essentially does not affect the integrity of the binder, the target, and the biological sample may be employed. In some embodiments, a chemical agent that does not affect the specificity of binding between the binder and the target may be employed.

In some embodiments, where two or more fluorophores may be employed simultaneously, a chemical agent may be capable of selectively modifying one or more signal generators. Susceptibility of different signal generators to a chemical agent may depend, in part, to the concentration of the signal generator, temperature, or pH. For example, two different fluorophores may have different susceptibility to a base depending upon the concentration of the base.

As used herein the term “brightfield type image” or “virtual stained image” (VSI) refers to an image of a biological sample that simulates that of an image obtained from a brightfield staining protocol. The image has similar contrast, intensity, and coloring as a brightfield image. This allows features within a biological sample, including but not limited to nuclei, epithelia, stroma or any type of extracellular matrix material features, to be characterized as if the brightfield staining protocol was used directly on the biological sample.

### Biological Samples

A biological sample in accordance with one embodiment of the invention may be solid or fluid. Biological sample refers to a sample obtained from a biological subject, including sample of biological tissue or fluid origin obtained in vivo or in vitro. Suitable examples of biological samples may include, but are not limited to, blood, saliva, cerebral spinal fluid, pleural fluid, milk, lymph, sputum, semen, urine, stool, tears, needle aspirates, external sections of the skin, respiratory, intestinal, and genitourinary tracts, tumors, organs, cell cultures, or solid tissue sections. In some embodiments, the biological sample may be analyzed as is, that is, without harvest and/or isolation of the target of interest. In an alternate embodiment, harvest of the sample may be performed prior to analysis. In some embodiments, the methods disclosed herein may be particularly suitable for in-vitro analysis of biological samples. Biological samples may be immobilized on a solid support, such as in glass slides, microtiter, or ELISA plates.

A biological sample may include any of the aforementioned samples regardless of their physical condition, such as, but not limited to, being frozen or stained or otherwise treated. In some embodiments, a biological sample may include compounds which are not naturally intermixed with the sample in nature such as preservatives, anticoagulants, buffers, fixatives, nutrients, antibiotics, or the like.

In some embodiments, a biological sample may include a tissue sample, a whole cell, a cell constituent, a cytospin, or a cell smear. In some embodiments, a biological sample essentially includes a tissue sample. A tissue sample may include a collection of similar cells obtained from a tissue of a biological subject that may have a similar function. In some embodiments, a tissue sample may include a collection of similar cells obtained from a tissue of a human. Suitable examples of human tissues include, but are not limited to, (1) epithelium; (2) the connective tissues, including blood vessels, bone and cartilage; (3) muscle tissue; and (4) nerve tissue. The source of the tissue sample may be solid tissue obtained from a fresh, frozen and/or preserved organ or tissue sample or biopsy or aspirate; blood or any blood constituents; bodily fluids such as cerebral spinal fluid, amniotic fluid, peritoneal fluid, or interstitial fluid; or cells from any time in gestation or development of the subject. In some embodiments, the tissue sample may include primary or cultured cells or cell lines.

The tissue sample may be obtained by a variety of procedures including, but not limited to surgical excision, aspiration or biopsy. The tissue may be fresh or frozen. In some embodiments, the tissue sample may be fixed and embedded in paraffin. The tissue sample may be fixed or otherwise preserved by conventional methodology; the choice of a fixative may be determined by the purpose for which the tissue is to be histologically stained or otherwise analyzed. The length of fixation may depend upon the size of the tissue sample and the fixative used. For example, neutral buffered formalin, Bouin's or paraformaldehyde, may be used to fix or preserve a tissue sample.

In some embodiments, a biological sample includes tissue sections of normal or cancerous origin, such as tissue sections form lymph node, colon, breast, prostate, lung, liver, and stomach. A tissue section may include a single part or piece of a tissue sample, for example, a thin slice of tissue or cells cut from a tissue sample. In some embodiments, multiple sections of tissue samples may be taken and subjected to analysis, provided the methods disclosed herein may be used for analysis of the same section of the tissue sample with respect to at least two different biomarkers. A tissue section, if employed as a biological sample may have a thickness in a range that is less than about 100 micrometers, in a range that is less than about 50 micrometers, in a range that is less than about 25 micrometers, or in range that is less than about 10 micrometers.

In certain embodiments, the biological samples are tissue samples suitable for the diagnosis of classical Hodgkin lymphoma. Such samples may come at least from lymph node biopsy, extranodal (mediastinal, retroperitoneal) biopsy, or bone marrow biopsy. The sample may be obtained by, for example, core needle biopsy, excisional biopsy or lymph node excision. The tissue sample may be fixed and embedded in paraffin using standard histology methods. Tissue sections are obtained from the tissue sample and used for the diagnostic methods according to embodiments of the invention. As described in the background section, RS/H cells are important for the diagnosis of classical Hodgkin lymphoma. RS/H cells are derived from B cells. The absence of RS/H cells has a very high negative predictive value for classical Hodgkin lymphoma. However, cells resembling RS/H cells are often associated with other diseases. Thus, it is important to be able to identify those RS/H cells that are truly Hodgkin lymphoma cells.

### Target or Biomarker of Interest

The target or biomarker of interest may include a target protein. A target protein according to an embodiment of the invention may be present on the surface of a biological sample (for example, an antigen on a surface of a tissue section) or present in the bulk of the sample (for example, an antibody in a buffer solution). In some embodiments, a target protein may not be inherently present on the surface of a biological sample and the biological sample may have to be processed to make the target protein available on the surface. In some embodiments, the target protein may be in a tissue, either on a cell surface, or within a cell.

Suitability of target protein to be analyzed may be determined by the type and nature of analysis required for the biological sample. In some embodiments, a target may provide information about the presence or absence of an analyte in the biological sample. In another embodiment, a target protein may provide information on a state of a biological sample. For example, if the biological sample includes a tissue sample, the methods disclosed herein may be used to detect target protein that may help in comparing different types of cells or tissues, comparing different developmental stages, detecting the presence of a disease or abnormality, or determining the type of disease or abnormality.

Suitable target proteins may include one or more of peptides, proteins (e.g., antibodies, affibodies, or aptamers), enzymes, ligands, receptors, antigens, or haptens. One or more of the aforementioned target proteins may be characteristic of particular cells, while other target proteins may be associated with a particular disease or condition. In some embodiments, target proteins in a tissue sample that may be detected and analyzed using the methods disclosed herein may include, but are not limited to, prognostic markers, predictive markers, hormone or hormone receptors, lymphoids, tumor markers, cell cycle associated markers, neural tissue and tumor markers, or cluster differentiation markers.

Suitable examples of prognostic markers may include enzymatic targets such as galactosyl transferase II, neuron specific enolase, proton ATPase-2, or acid phosphatase. Other examples of prognostic protein or gene markers include Ki67, cyclin E, p53, cMet.

Suitable examples of predictive markers (drug response) may include protein or gene targets such as EGFR, Her2, ALK.

Suitable examples of hormone or hormone receptors may include human chorionic gonadotropin (HCG), adrenocorticotropic hormone, carcinoembryonic antigen (CEA), prostate-specific antigen (PSA), estrogen receptor, progesterone receptor, androgen receptor, gClq-R/p33 complement receptor, IL-2 receptor, p75 neurotrophin receptor, PTH receptor, thyroid hormone receptor, or insulin receptor.

Suitable examples of lymphoid markers may include alpha-1-antichymotrypsin, alpha-1-antitrypsin, B cell target, bcl-2, bcl-6, B lymphocyte antigen 36 kD, BM1 (myeloid target), BM2 (myeloid target), galectin-3, granzyme B, HLA class I Antigen, HLA class II (DP) antigen, HLA class II (DQ) antigen, HLA class II (DR) antigen, human neutrophil defensins, immunoglobulin A, immunoglobulin D, immunoglobulin G, immunoglobulin M, kappa light chain, kappa light chain, lambda light chain, lymphocyte/histocyte antigen, macrophage target, muramidase (lysozyme), p80 anaplastic lymphoma kinase, plasma cell target, secretory leukocyte protease inhibitor, T cell antigen receptor (JOVI 1), T cell antigen receptor (JOVI 3), terminal deoxynucleotidyl transferase, or unclustered B cell target.

Suitable examples of tumor markers may include alpha fetoprotein, apolipoprotein D, BAG-1 (RAP46 protein), CA19-9 (sialyl lewisa), CA50 (carcinoma associated mucin antigen), CAl25 (ovarian cancer antigen), CA242 (tumour associated mucin antigen), chromogranin A, clusterin (apolipoprotein J), epithelial membrane antigen, epithelial-related antigen, epithelial specific antigen, gross cystic disease fluid protein-15, hepatocyte specific antigen, heregulin, human gastric mucin, human milk fat globule, MAGE-1, matrix metalloproteinases, melan A, melanoma target (HMB45), mesothelin, metallothionein, microphthalmia transcription factor (MITF), Muc-1 core glycoprotein. Muc-1 glycoprotein, Muc-2 glycoprotein, Muc-5AC glycoprotein, Muc-6 glycoprotein, myeloperoxidase, Myf-3 (Rhabdomyosarcoma target), Myf-4 (Rhabdomyosarcoma target), MyoD1 (Rhabdomyosarcoma target), myoglobin, nm23 protein, placental alkaline phosphatase, prealbumin, prostate specific antigen, prostatic acid phosphatase, prostatic inhibin peptide, PTEN, renal cell carcinoma target, small intestinal mucinous antigen, tetranectin, thyroid transcription factor-1, tissue inhibitor of matrix metalloproteinase 1, tissue inhibitor of matrix metalloproteinase 2, tyrosinase, tyrosinase-related protein-1, villin, or von Willebrand factor.

Suitable examples of cell cycle associated markers may include apoptosis protease activating factor-1, bcl-w, bcl-x, bromodeoxyuridine, CAK (cdk-activating kinase), cellular apoptosis susceptibility protein (CAS), caspase 2, caspase 8, CPP32 (caspase-3), CPP32 (caspase-3), cyclin dependent kinases, cyclin A, cyclin B1, cyclin D1, cyclin D2, cyclin D3, cyclin E, cyclin G, DNA fragmentation factor (N-terminus), Fas (CD95), Fas-associated death domain protein, Fas ligand, Fen-1, IPO-38, Mcl-1, minichromosome maintenance proteins, mismatch repair protein (MSH2), poly (ADP-Ribose) polymerase, proliferating cell nuclear antigen, p16 protein, p27 protein, p34cdc2, p57 protein (Kip2), p105 protein, Stat 1 alpha, topoisomerase I, topoisomerase II alpha, topoisomerase III alpha, or topoisomerase II beta.

Suitable examples of cluster differentiation markers may include CD1a, CD1b, CD1c, CD1d, CD1e, CD2, CD3delta, CD3epsilon, CD3gamma, CD4, CD5, CD6, CD7, CD8alpha, CD8beta, CD9, CD10, CD11a, CD11b, CD11c, CDw12, CD13, CD14, CD15, CD15s, CD16a, CD16b, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD44R, CD45, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD65s, CD66a, CD66b, CD66c, CD66d, CD66e, CD66f, CD68, CD69, CD70, CD71, CD72, CD73, CD74, CDw75, CDw76, CD77, CD79A, CD79B, CD80, CD81, CD82, CD83, CD84, CD85, CD86, CD87, CD88, CD89, CD90, CD91, CDw92, CDw93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CDw119, CD120a, CD120b, CD121a, CDw121b, CD122, CD123, CD124, CDw125, CD126, CD127, CDw128a, CDw128b, CD130, CDw131, CD132, CD134, CD135, CDw136, CDw137, CD138, CD139, CD140a, CD140b, CD141, CD142, CD143, CD144, CDw145, CD146, CD147, CD148, CDw149, CDw150, CD151, CD152, CD153, CD154, CD155, CD156, CD157, CD158a, CD158b, CD161, CD162, CD163, CD164, CD165, CD166, and TCR-zeta.

Other suitable target proteins include centromere protein-F (CENP-F), giantin, involucrin, lamin A&C (XB 10), LAP-70, mucin, nuclear pore complex proteins, p180 lamellar body protein, ran, cathepsin D, Ps2 protein, Her2-neu, P53, S100, epithelial target antigen (EMA), TdT, MB2, MB3, PCNA, Ki67, cytokeratin, PI3K, cMyc or MAPK.

Still other suitable target proteins include Her2/neu (epidermal growth factor over expressed in breast and stomach cancer, therapy by a monoclonal antibody slows tumor growth); EGF-R/erbB (epidermal growth factor receptor); ER (estrogen receptor required for growth of some breast cancer tumors, located in the nucleus and detected with ISH for deciding on therapy limiting estrogen in positive patients); PR (progesterone receptor is a hormone that binds to DNA); AR (androgen receptor is involved in androgen dependent tumor growth); β-catenin (oncogene in cancer translocates from the cell membrane to the nucleus, which functions in both cell adhesion and as a latent gene regulatory protein); Phospho-β-Catenin: phosphorylated (form of β-catenin degrades in the cytosol and does not translocate to the nucleus); GSK3β (glycogen synthase kinase-3β protein in the Wnt pathway phosphorylates β-catenin marking the phospo-β-catenin for rapid degradation in the protostomes); PKCβ (mediator G-protein coupled receptor); NFKβ (nuclear factor kappa B marker for inflammation when translocated to the nucleus); VEGF (vascular endothelial growth factor related to angiogenesis); E-cadherin (cell to cell interaction molecule expressed on epithelial cells, the function is lost in epithelial cancers); c-met (tyrosine kinase receptor).

In certain embodiments, the target or biomarker of interest is a polysaccharide antigen.

In certain embodiments, the target or biomarker of interest includes biomarkers informative for the diagnosis of classical Hodgkin lymphoma, as compared to other lymphocyte diseases. Exemplary biomarkers useful for the diagnosis of classical Hodgkin lymphoma include CD30, CD15, CD45, PAX-5, CD20, CD3, CD79A, BOB1 and OCT-2. Exemplary biomarkers useful for the diagnosis of classical Hodgkin lymphoma also include MUM-1, Fascin, EBV LMP-1, BCL-6, CD138, EMA, cytotoxic markers, ALK, CD43 and other T-cell markers, kappa/lambda, etc. Another biomarker helpful in the assessment of classical Hodgkin lymphoma is EBER.

CD30 is a cytokine receptor belonging to TNF superfamily, expressed in RS/H cells and other cells. CD30 stains in a paranuclear pattern along with a membranous distribution. CD30 stains positive in about 99% of classical Hodgkin lymphoma cases.

By CD15 it is meant the CD15 carbohydrate antigen carried by both glycoproteins and glycolipids. This carbohydrate antigen is also called 3-fucosyl-N-acetyl-lactosamine, Lewis x antigen, Lewis x, LeX, X-hapten, X-antigen or SSEA-1 (stage-specific embryonic antigen 1). It is produced by, among other enzymes, the CD15/FUT4 protein. CD15 stains positive in about 85% of classical Hodgkin lymphoma cases.

CD45 is a transmembrane protein involved in co-stimulation of differentiated hematopoietic cells, expressed in many T cells but not typically in RS/H cells involved in classical Hodgkin lymphoma. The protein encoded by this gene is a member of the protein tyrosine phosphatase (PTP) family. CD45 consists of multiple isoforms (e.g., CD45RA, CD45RB, CD45R0) that are all products of a single complex gene. The different CD45 isoforms are expressed on different hematopoietic cell types, and have different epitopes. For classical Hodgkin lymphoma, CD45RB and or pan-CD45 antibodies are useful because these antibodies stain most hematopoietic cell types, including B- and T-cells. CD45 stains positive in about 10-15% of classical Hodgkin lymphoma cases.

Pax-5 is used for detecting nuclei of B cells and RS/H cells involved in classical Hodgkin lymphoma, the latter typically showing a weaker nuclear staining than the former. PAX-5 is weakly positive in 90-95% of cases.

CD3 is virtually always negative in classical Hodgkin lymphoma, nodular lymphocyte predominant Hodgkin lymphoma, and B-cell lymphoma, and is usually (but not always) positive in T-cell lymphomas. So, CD3 positivity in Hodgkin-like cells will essentially rule out the possibility of classical Hodgkin lymphoma. In certain embodiments, detection of the CD3-TCR complex may be achieved by detecting any of the subunits CD3delta, CD3epsilon, CD3gamma, or CD3zeta. In a more preferred embodiment, the detection of the CD3-TCR complex is achieved by detecting the subunit CD3epsilon.

CD20 is usually expressed in about 20-25% of cases of Hodgkin lymphoma, usually showing a variable staining pattern (some cells negative, some cells weak, some cells stronger).

Other biomarkers helpful in the assessment of classical Hodgkin lymphoma also include CD79A, BOB1, OCT-2, etc. CD79A, BOB1, and OCT-2 are each expressed in about 15% of cases of classical Hodgkin lymphoma. Some literature suggests that their expression is independent of one another.

Still other biomarkers helpful in the assessment of classical Hodgkin lymphoma and their respective expression level include MUM-1 (98%), Fascin (90%), EBV LMP-1 (30-40%), BCL-6 (40%), CD138 30%), EMA 5%), cytotoxic markers (<5%), ALK (negative), CD43 and other T-cell markers (negative), kappa/lambda (negative), etc.

There are many other markers that could be used, depending on the specific differential diagnosis and the preferences and experience of the particular pathologist. Nonetheless, the key in a correct diagnosis lies in the overall phenotype in context of the morphologic findings.

RS/H cells indicative of classical Hodgkin lymphoma are often CD30+, CD15+, CD45-, CD3- and Pax-5+. If these results are not clear cut (whether due to overlapping results or hard to interpret results), the results from CD79a, OCT-1, and BOB1 provides further information for a definitive diagnosis. Additional common hematolymphoid markers useful for the detection of Hodgkin cells of classical Hodgkin lymphoma include MUM-1 (98%+), Fascin (90%+), EBV LMP-1 (30-40%+), BCL-6 (40%+), CD138 (30%+), EMA (<5%+), Cytotoxic markers (<5%+), ALK (0%), CD43 and other T-cell markers (?0%), as well as Kappa/lambda (both are negative in classical Hodgkin lymphoma, but often show a polytypic non-specific blush due to non-specific uptake).

Subsets of these markers may be useful in specific differential diagnostic situations. For example T-cell (CD2, CD3, CD43)/cytotoxic (granzyme B, perforin, TIA-1) markers and ALK may be useful in the differential diagnosis with anaplastic large cell lymphoma since they are almost always negative in classical Hodgkin lymphoma but frequently positive in anaplastic large cell lymphoma. MUM-1, in combination with BOB1, OCT-2, and CD79a, may be useful in the differential diagnosis with T/histiocyte-rich B-cell lymphoma or nodular lymphocyte predominance Hodgkin lymphoma, as MUM-1 is almost always positive in classical Hodgkin lymphoma, and almost always negative in the nodular lymphocyte predominance Hodgkin lymphoma. LMP-1 or EBER may be useful in the differential diagnosis of classical Hodgkin lymphoma, particularly in children and the elderly, in which EBV-positivity is much more common.

Some markers have prognostic significance in classical Hodgkin lymphoma. For example the number of CD68+ host cells (i.e., non-neoplastic reactive cells that are present) predicts a poor prognosis in Hodgkin patient, while expression of CD20 or lack of expression of CD15 on the Hodgkin cells is correlated with poor prognosis. EBV positivity on the Hodgkin cells in patients older than 60, and EBV negativity in patients younger than 15 also predicts a poor prognosis.

The differential diagnosis of classical Hodgkin lymphoma with non-hematolymphoid neoplasms may be difficult. Pancytokeratin (expressed in carcinoma) may be helpful in its distinction from carcinoma, S-100 (expressed in melanoma) may be helpful in its distinction from melanoma, while, SALL4 and OCT4 (expressed in germ cells tumors and embryonal carcinoma/seminoma) may be helpful in its distinction from germ cell tumors.

There is a lymphoma intermediate between classical Hodgkin lymphoma and diffuse large B-cell lymphoma. The latter is characterized by frequent expression of CD30, CD15, CD45, CD20, CD79a, PAX-5, OCT-2, and BOB1, infrequent expression of bcl-6 and CD10, and negativity for ALK and T-cell/cytotoxic markers.

The differential diagnosis of classical Hodgkin lymphoma and T-cell histiocyte-rich B-cell lymphoma may be realized by the expression pattern of several biomarkers. The latter is characterized by frequent expression of CD45, CD20, CD79a, OCT-2, and BOB1, strong and frequent expression of PAX-5, and infrequent expression of CD30 (5%), CD15 (1%) and EBV LMP-1 (1%).

The differential diagnosis of classical Hodgkin lymphoma and peripheral T-cell lymphoma may be realized again by the expression pattern of several biomarkers. The latter is characterized by frequent expression of CD45 and CD3 and other T-cell markers, variable expression of CD30, infrequent expression of CD15 (<5%), PAX-5 (<5%) and EBV LMP-1 (1%).

The differential diagnosis of classical Hodgkin lymphoma and diffuse large B-cell lymphoma may be realized by the expression pattern of several biomarkers. CD138 is typically negative in Hodgkin lymphoma (only positive in 30% of cases), but is often positive in plasmablastic variants of diffuse large B-cell lymphoma. Classical Hodgkin lymphoma and anaplastic large cell lymphoma both are CD30-positive. However, cases of anaplastic large cell lymphoma are often positive for CD45, CD3, CD43, EMA, cytotoxic markers, and ALK, while cases of classical lymphoma are usually negative for these markers, but usually positive for PAX-5, CD15 and sometimes positive for EBV-LMP and EBER, as well as CD20. Markers useful in the distinction of classical Hodgkin cells and sarcomas include CD30 and CD15. Both markers are usually positive on classical Hodgkin cells, but negative in pleomorphic sarcomas.

The lymphocyte-rich variant of classical Hodgkin lymphoma has a similar phenotype to other forms of classical Hodgkin lymphoma, but may have a higher incidence of the B-cell related markers CD79a, OCT-2, and BOB1. It may also be associated with EBV positivity. Its differential diagnosis specifically includes nodular lymphocyte predominance Hodgkin lymphoma (see next paragraph for expression pattern), small lymphocytic lymphoma/chronic lymphocytic leukemia (small lymphocytic lymphoma/chronic lymphocytic leukemia is usually positive for CD20, CD43, CD45, CD5, CD23, and negative for CD15, CD30, and EBV-LMP-1) and reactive interfollicular hyperplasia (reactive interfollicular hyperplasia usually has variable numbers of cells positive for CD20, CD3, and CD30 (often with a variation in the intensity of CD30 staining), and is usually negative for CD15 and EBV-LMP-1).

Nodular lymphocyte predominance Hodgkin lymphoma represents about 5% of cases of Hodgkin lymphoma. It can be distinguished from classical Hodgkin lymphoma by its expression of CD45 and the B-lineage markers CD20, CD79a, PAX-5, BOB1, and OCT2, along with weak or negative staining for CD30 and CD15. Other informative markers and expression patterns include the expression of bcl-2 (95%), EMA (70%) and lack of expression of CD3, CD43, CD10 and MUM-1. In addition, there is often a ring of CD57/PD-1/bcl-6 positive “host” cells around the LP cells, the type of large, atypical cell present in nodular lymphocyte predominance Hodgkin lymphoma. OCT-2 can be very useful in demonstrating the LP cells, since they typically stain very strongly.

In certain embodiments, the target or biomarker of interest includes biomarkers that may be detected by antibodies against each of CD30, CD 15, CD45, PAX-5, CD20, CD3, CD79A, BOB1 and OCT-2. Exemplary antibodies are:

Monoclonal Mouse Anti-Human CD3 Clone F7.2.38 (Dako),

Monoclonal Rabbit Anti-Human CD3 Clone SP7 (Spring Biosciences),

Monoclonal Mouse Anti-Human CD3 Clone SP162 (Spring Biosciences),

Monoclonal Mouse Anti-Human CD45, Leucocyte Common Antigen, Clones 2B11+PD7/26 (Dako),

Monoclonal Rabbit Anti-Human PAX5, Clone EP156 (Epitomics),

Polyclonal Rabbit Anti-Human BOB1, Clone sc-955 (Santa Cruz),

Polyclonal Rabbit Anti-Human Oct-2, Clone sc-233 (Santa Cruz),

Monoclonal Rabbit Anti-Human CD20 (Epitomics Catalogue number 1632-X),

Monoclonal mouse Anti-Human Granulocyte-Associated Ant, CD15, Clone C3D-1 (Dako),

Monoclonal mouse Anti-Human CD790c, Clone JCB117 (Dako), and Monoclonal mouse Anti-Human CD30, Clone 1G12 (Leica).

The representative polypeptide sequences of these proteins are listed below. Nonetheless, alternative isoforms (derived from alternative transcripts of the gene of interest) are also encompassed by the present application.

CD30 protein sequence (SEQ ID NO: 1):
MRVLLAALGLLFLGALRAFPQDRPFEDTCHGNPSHYYDKAVRRCCYRCP
MGLFPTQQCPQRPTDCRKQCEPDYYLDEADRCTACVTCSRDDLVEKTPC
AWNSSRVCECRPGMFCSTSAVNSCARCFFHSVCPAGMIVKFPGTAQKNT
VCEPASPGVSPACASPENCKEPSSGTIPQAKPTPVSPATSSASTMPVRG
GTRLAQEAASKLTRAPDSPSSVGRPSSDPGLSPTQPCPEGSGDCRKQCE
PDYYLDEAGRCTACVSCSRDDLVEKTPCAWNSSRTCECRPGMICATSAT
NSRARCVPYPICAAETVTKPQDMAEKDTTFEAPPLGTQPDCNPTPENGE
APASTSPTQSLLVDSQASKTLPIPTSAPVALSSTGKPVLDAGPVLFWVI
LVLVVVVGSSAFLLCHRRACRKRIRQKLHLCYPVQTSQPKLELVDSRPR
RSSTQLRSGASVTEPVAEERGLMSQPLMETCHSVGAAYLESLPLQDASP
AGGPSSPRDLPEPRVSTEHTNNKIEKIYIMKADTVIVGTVKAELPEGRG
LAGPAEPELEEELEADHTPHYPEQETEPPLGSCSDVMLSVEEEGKEDPL
PTAASGK
CD15/FUT4 protein sequence (SEQ ID NO: 2):
MRRLWGAARKPSGAGWEKEWAEAPQEAPGAWSGRLGPGRSGRKGRAVPG
WASWPAHLALAARPARHLGGAGQGPRPLHSGTAPFHSRASGERQRRLEP
QLQHESRCRSSTPADAWRAEAALPVRAMGAPWGSPTAAAGGRRGWRRGR
GLPWTVCVLAAAGLTCTALITYACWGQLPPLPWASPTPSRPVGVLLWWE
PFGGRDSAPRPPPDCRLRFNISGCRLLTDRASYGEAQAVLFHHRDLVKG
PPDWPPPWGIQAHTAEEVDLRVLDYEEAAAAAEALATSSPRPPGQRWVW
MNFESPSHSPGLRSLASNLFNWTLSYRADSDVFVPYGYLYPRSHPGDPP
SGLAPPLSRKQGLVAWVVSHWDERQARVRYYHQLSQHVTVDVFGRGGPG
QPVPEIGLLHTVARYKFYLAFENSQHLDYITEKLWRNALLAGAVPVVLG
PDRANYERFVPRGAFIHVDDFPSASSLASYLLFLDRNPAVYRRYFHWRR
SYAVHITSFWDEPWCRVCQAVQRAGDRPKSIRNLASWFER
CD20 protein sequence (SEQ ID NO: 3):
MTTPRNSVNGTFPAEPMKGPIAMQSGPKPLFRRMSSLVGPTQSFFMRES
KTLGAVQIMNGLFHIALGGLLMIPAGIYAPICVTVWYPLWGGIMYIISG
SLLAATEKNSRKCLVKGKMIMNSLSLFAAISGMILSIMDILNIKISHFL
KMESLNFIRAHTPYINIYNCEPANPSEKNSPSTQYCYSIQSLFLGILSV
MLIFAFFQELVIAGIVENEWKRTCSRPKSNIVLLSAEEKKEQTIEIKEE
VVGLTETSSQPKNEEDIEIIPIQEEEEEETETNFPEPPQDQESSPIEND
SSP
Pax-5 protein sequence (SEQ ID NO: 4):
MDLEKNYPTPRTSRTGHGGVNQLGGVFVNGRPLPDVVRQRIVELAHQGV
RPCDISRQLRVSHGCVSKILGRYYETGSIKPGVIGGSKPKVATPKVVEK
IAEYKRQNPTMFAWEIRDRLLAERVCDNDTVPSVSSINRIIRTKVQQPP
NQPVPASSHSIVSTGSVTQVSSVSTDSAGSSYSISGILGITSPSADTNK
RKRDEGIQESPVPNGHSLPGRDFLRKQMRGDLFTQQQLEVLDRVFERQH
YSDIFTTTEPIKPEQTTEYSAMASLAGGLDDMKANLASPTPADIGSSVP
GPQSYPIVTGRDLASTTLPGYPPHVPPAGQGSYSAPTLTGMVPGSEFSG
SPYSHPQYSSYNDSWRFPNPGLLGSPYYYSAAARGAAPPAAATAYDRH
CD45 protein sequence (SEQ ID NO: 5):
MTMYLWLKLLAFGFAFLDTEVFVTGQSPTPSPTGLTTAKMPSVPLSSDP
LPTHTTAFSPASTFERENDFSETTTSLSPDNTSTQVSPDSLDNASAFNT
TGVSSVQTPHLPTHADSQTPSAGTDTQTFSGSAANAKLNPTPGSNAISD
VPGERSTASTFPTDPVSPLTTTLSLAHHSSAALPARTSNTTITANTSDA
YLNASETTTLSPSGSAVISTTTIATTPSKPTCDEKYANITVDYLYNKET
KLFTAKLNVNENVECGNNTCTNNEVHNLTECKNASVSISHNSCTAPDKT
LILDVPPGVEKFQLHDCTQVEKADTTICLKWKNIETFTCDTQNITYRFQ
CGNMIFDNKEIKLENLEPEHEYKCDSEILYNNHKFTNASKIIKTDFGSP
GEPQIIFCRSEAAHQGVITWNPPQRSFHNFTLCYIKETEKDCLNLDKNL
IKYDLQNLKPYTKYVLSLHAYIIAKVQRNGSAAMCHFTTKSAPPSQVWN
MTVSMTSDNSMHVKCRPPRDRNGPHERYHLEVEAGNTLVRNESHKNCDF
RVKDLQYSTDYTFKAYFHNGDYPGEPFILHHSTSYNSKALIAFLAFLII
VTSIALLVVLYKIYDLHKKRSCNLDEQQELVERDDEKQLMNVEPIHADI
LLETYKRKIADEGRLFLAEFQSIPRVFSKFPIKEARKPFNQNKNRYVDI
LPYDYNRVELSEINGDAGSNYINASYIDGFKEPRKYIAAQGPRDETVDD
FWRMIWEQKATVIVMVTRCEEGNRNKCAEYWPSMEEGTRAFGDVVVKIN
QHKRCPDYIIQKLNIVNKKEKATGREVTHIQFTSWPDHGVPEDPHLLLK
LRRRVNAFSNFFSGPIVVHCSAGVGRTGTYIGIDAMLEGLEAENKVDVY
GYVVKLRRQRCLMVQVEAQYILIHQALVEYNQFGETEVNLSELHPYLHN
MKKRDPPSEPSPLEAEFQRLPSYRSWRTQHIGNQEENKSKNRNSNVIPY
DYNRVPLKHELEMSKESEHDSDESSDDDSDSEEPSKYINASFIMSYWKP
EVMIAAQGPLKETIGDFWQMIFQRKVKVIVMLTELKHGDQEICAQYWGE
GKQTYGDIEVDLKDTDKSSTYTLRVFELRHSKRKDSRTVYQYQYTNWSV
EQLPAEPKELISMIQVVKQKLPQKNSSEGNKHHKSTPLLIHCRDGSQQT
GIFCALLNLLESAETEEVVDIFQVVKALRKARPGMVSTFEQYQFLYDVI
ASTYPAQNGQVKKNNHQEDKIEFDNEVDKVKQDANCVNPLGAPEKLPEA
KEQAEGSEPTSGTEGPEHSVNGPASPALNQGS
CD3, delta (CD3-TCR complex) protein sequence
(SEQ ID NO: 6):
MEHSTFLSGLVLATLLSQVSPFKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK
CD3, epsilon (CD3-TCR complex) protein sequence
(SEQ ID NO: 7):
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVC
YPRGSKPEDANFYLYLRARVCENCMEMDVMSVATIVIVDICITGGLLLL
VYYWSKNRKAKAKPVTRGAGAGGRQRGQNKERPPPVPNPDYEPIRKGQR
DLYSGLNQRRI
CD3, gamma (CD3-TCR complex) protein sequence
(SEQ ID NO: 8):
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN
CD3, zeta subunit protein sequence (SEQ ID NO: 9):
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR
CD79A protein sequence (SEQ ID NO: 10):
MPGGPGVLQALPATIFLLFLLSAVYLGPGCQALWMHKVPASLMVSLGED
AHFQCPHNSSNNANVTWWRVLHGNYTWPPEFLGPGEDPNGTLIIQNVNK
SHGGIYVCRVQEGNESYQQSCGTYLRVRQPPPRPFLDMGEGTKNRIITA
EGIILLFCAVVPGTLLLFRKRWQNEKLGLDAGDEYEDENLYEGLNLDDC
SMYEDISRGLQGTYQDVGSLNIGDVQLEKP
OCT2 protein sequence (SEQ ID NO: 11):
MVHSSMGAPEIRMSKPLEAEKQGLDSPSEHTDTERNGPDTNHQNPQNKT
SPFSVSPTGPSTKIKAEDPSGDSAPAAPLPPQPAQPHLPQAQLMLTGSQ
LAGDIQQLLQLQQLVLVPGHHLQPPAQFLLPQAQQSQPGLLPTPNLFQL
PQQTQGALLTSQPRAGLPTQAVTRPTLPDPHLSHPQPPKCLEPPSHPEE
PSDLEELEQFARTFKQRRIKLGFTQGDVGLAMGKLYGNDFSQTTISRFE
ALNLSFKNMCKLKPLLEKWLNDAETMSVDSSLPSPNQLSSPSLGFDGLP
GRRRKKRTSIETNVRFALEKSFLANQKPTSEEILLIAEQLHMEKEVIRV
WFCNRRQKEKRINPCSAAPMLPSPGKPASYSPHMVTPQGGAGTLPLSQA
SSSLSTTVTTLSSAVGTLHPSRTAGGGGGGGGAAPPLNSIPSVTPPPPA
TTNSTNPSPQGSHSAIGLSGLNPSTGPGLWWNPAPYQP
BOB1 protein sequence (SEQ ID NO: 12):
MLWQKPTAPEQAPAPARPYQGVRVKEPVKELLRRKRGHASSGAAPAPTA
VVLPHQPLATYTTVGPSCLDMEGSVSAVTEEAALCAGWLSQPTPATLQP
LAPWTPYTEYVPHEAVSCPYSADMYVQPVCPSYTVVGPSSVLTYASPPL
ITNVTTRSSATPAVGPPLEGPEHQAPLTYFPWPQPLSTLPTSTLQYQPP
APALPGPQFVQLPISIPEPVLQDMEDPRRAASSLTIDKLLLEEEDSDAY
ALNHTLSVEGF

The target or biomarker of interest may include a target nucleic acid. A target nucleic acid sequence according to an embodiment of the invention refers to a sequence of interest which is contained in a nucleic acid molecule in the biological sample. The nucleic acid molecule may be present in the nuclei of the cells of the biological sample (for example, chromosomal DNA) or present in the cytoplasm (for example, mRNA). In some embodiments, a nucleic acid molecule may not be inherently present on the surface of a biological sample and the biological sample may have to be processed to make the nucleic acid molecule accessible by a probe. For example, protease treatment of the sample could readily expose the target nucleic acid sequences.

Suitability of a nucleic acid molecule to be analyzed may be determined by the type and nature of analysis required for the biological sample. In some embodiments, the analysis may provide information about the gene expression level of the target nucleic acid sequence in the biological sample. In other embodiments, the analysis may provide information on the presence or absence or amplification level of a chromosomal DNA. For example, if the biological sample includes a tissue sample, the methods disclosed herein may be used to detect a target nucleic acid sequence that may identify cells which has an increased copy number of a particular chromosomal segment harboring the target nucleic acid sequence. Alternatively, the methods may be used to detect cells which have an increased copy number of all the chromosomes (hyperploidy).

In some embodiments, the target nucleic acid sequence in a tissue sample that may be detected and analyzed using the methods disclosed herein may include, but are not limited to, nucleic acid sequences for prognostic markers, hormone or hormone receptors, lymphoids, tumor markers, cell cycle associated markers, neural tissue and tumor markers, or cluster differentiation markers.

In certain embodiments, the target nucleic acid sequence includes a sequence that is part of the gene sequence which encodes the target protein disclosed above. In other embodiments, the target nucleic acid sequence does not include a sequence that is part of the gene sequence which encodes the target protein. Thus, the target nucleic acid sequence may include a sequence that is part of the gene sequence which encodes a different protein than the target protein, or a sequence that identifies other features of a chromosome (e.g., centromere sequence).

### Binders

The methods disclosed herein involve the use of binders that physically bind to the target in a specific manner. In some embodiments, a binder may bind to a target with sufficient specificity, that is, a binder may bind to a target with greater affinity than it does to any other molecule. In some embodiments, the binder may bind to other molecules, but the binding may be such that the non-specific binding may be at or near background levels. In some embodiments, the affinity of the binder for the target of interest may be in a range that is at least 2-fold, at least 5-fold, at least 10-fold, or more than its affinity for other molecules. In some embodiments, binders with the greatest differential affinity may be employed, although they may not be those with the greatest affinity for the target.

Binding between the target and the binder may be affected by physical binding. Physical binding may include binding effected using non-covalent interactions. Non-covalent interactions may include, but are not limited to, hydrophobic interactions, ionic interactions, hydrogen-bond interactions, or affinity interactions (such as, biotin-avidin or biotin-streptavidin complexation). In some embodiments, the target and the binder may have areas on their surfaces or in cavities giving rise to specific recognition between the two resulting in physical binding. In some embodiments, a binder may bind to a biological target based on the reciprocal fit of a portion of their molecular shapes.

Binders and their corresponding targets may be considered as binding pairs, of which non-limiting examples include immune-type binding-pairs, such as, antigen/antibody, antigen/antibody fragment, or hapten/anti-hapten; nonimmune-type binding-pairs, such as biotin/avidin, biotin/streptavidin, folic acid/folate binding protein, hormone/hormone receptor, lectin/specific carbohydrate, enzyme/enzyme, enzyme/substrate, enzyme/substrate analog, enzyme/pseudo-substrate (substrate analogs that cannot be catalyzed by the enzymatic activity), enzyme/co-factor, enzyme/modulator, enzyme/inhibitor, or vitamin B12/intrinsic factor. Other suitable examples of binding pairs may include complementary nucleic acid fragments (including DNA sequences, RNA sequences, PNA sequences, and peptide nucleic acid sequences, locked nucleic acid sequences); Protein A/antibody; Protein G/antibody; nucleic acid/nucleic acid binding protein; or polynucleotide/polynucleotide binding protein.

In some embodiments, the binder may be a sequence- or structure-specific binder, wherein the sequence or structure of a target recognized and bound by the binder may be sufficiently unique to that target.

In some embodiments, the binder may be structure-specific and may recognize a primary, secondary, or tertiary structure of a target. A primary structure of a target may include specification of its atomic composition and the chemical bonds connecting those atoms (including stereochemistry), for example, the type and nature of linear arrangement of amino acids in a protein. A secondary structure of a target may refer to the general three-dimensional form of segments of biomolecules, for example, for a protein a secondary structure may refer to the folding of the peptide “backbone” chain into various conformations that may result in distant amino acids being brought into proximity with each other. Suitable examples of secondary structures may include, but are not limited to, alpha helices, beta pleated sheets, or random coils. A tertiary structure of a target may be is its overall three dimensional structure. A quaternary structure of a target may be the structure formed by its noncovalent interaction with one or more other targets or macromolecules (such as protein interactions). An example of a quaternary structure may be the structure formed by the four-globin protein subunits to make hemoglobin. A binder in accordance with the embodiments of the invention may be specific for any of the afore-mentioned structures.

An example of a structure-specific binder may include a protein-specific molecule that may bind to a protein target. Examples of suitable protein-specific molecules may include antibodies and antibody fragments, nucleic acids (for example, aptamers that recognize protein targets), or protein substrates (non-catalyzable).

In some embodiments, a target may include an antigen and a binder may include an antibody. A suitable antibody may include monoclonal antibodies, polyclonal antibodies, multispecific antibodies (for example, bispecific antibodies), or antibody fragments so long as they bind specifically to a target antigen.

In some embodiments, a target may include a monoclonal antibody. A “monoclonal antibody” may refer to an antibody obtained from a population of substantially homogeneous antibodies, that is, the individual antibodies comprising the population are identical except for possible naturally occurring mutations that may be present in minor amounts. Monoclonal antibodies may be highly specific, being directed against a single antigenic site. Furthermore, in contrast to (polyclonal) antibody preparations that typically include different antibodies directed against different determinants (epitopes), each monoclonal antibody may be directed against a single determinant on the antigen. A monoclonal antibody may be prepared by any known method such as the hybridoma method, by recombinant DNA methods, or may be isolated from phage antibody libraries.

In a specific embodiment, the binders are antibodies that each bind to an antigen particularly suited for the diagnosis of classic Hodgkin lymphoma. Exemplary antibodies include antibody for each of CD30, CD45, CD15, CD3, CD20, Pax-5, CD79A, BOB1 and OCT-2, respectively. Other antibodies that bind to an antigen particularly suited for the diagnosis of classic Hodgkin lymphoma include antibody for each of MUM-1, Fascin, EBV LMP-1, BCL-6, CD138, EMA, cytotoxic markers, ALK, CD43 and other T-cell markers, kappa/lambda, etc.

In some embodiments, a biological sample may include a cell or a tissue sample and the methods disclosed herein may be employed in immunofluorescence (IF). Immunochemistry may involve binding of a target antigen to an antibody-based binder to provide information about the tissues or cells (for example, diseased versus normal cells).

Regardless of the type of binder and the target, the specificity of binding between the binder and the target may also be affected depending on the binding conditions (for example, hybridization conditions in case of complementary nucleic acids). Suitable binding conditions may be realized by modulating one or more of pH, temperature, or salt concentration.

As noted hereinabove, a binder may be intrinsically labeled (fluorophore attached during synthesis of binder) with a fluorophore or extrinsically labeled (fluorophore attached during a later step). For example for a protein-based binder, an intrinsically labeled binder may be prepared by employing fluorophore labeled amino acids. In some embodiments, a binder may be synthesized in a manner such that fluorophore may be incorporated at a later stage. In some embodiments, a binder such as a protein (for example, an antibody) or a nucleic acid (for example, a DNA) may be directly chemically labeled using appropriate chemistries for the same.

In some embodiments, combinations of binders may be used that may provide greater specificity or in certain embodiments amplification of the signal. Thus, in some embodiments, a sandwich of binders may be used, where the first binder may bind to the target and serve to provide for secondary binding, where the secondary binder may or may not include a fluorophore, which may further provide for tertiary binding (if required) where the tertiary binding member may include a fluorophore.

In some embodiments, signal amplification may be obtained when several secondary antibodies may bind to epitopes on the primary antibody. In an immunofluorescence procedure a primary antibody may be the first antibody used in the procedure and the secondary antibody may be the second antibody used in the procedure. In some embodiments, a primary antibody may be the only antibody used in an IF procedure.

In some embodiments, a probe is used to detect the target nucleic acid sequences. It is desirable that the probe binds specifically to the region of nucleic acid molecule that contains the sequence of interest. Thus, in some embodiments, the probe is sequence-specific. A sequence-specific probe may include a nucleic acid and the probe may be capable of recognizing a particular linear arrangement of nucleotides or derivatives thereof. In some embodiments, the linear arrangement may include contiguous nucleotides or derivatives thereof that may each bind to a corresponding complementary nucleotide in the probe. In an alternate embodiment, the sequence may not be contiguous as there may be one, two, or more nucleotides that may not have corresponding complementary residues on the probe. Suitable examples of probes may include, but are not limited to DNA or RNA oligonucleotides or polynucleotides, peptide nucleic acid (PNA) sequences, locked nucleic acid (LNA) sequences, or aptamers. In some embodiments, suitable probes may include nucleic acid analogs, such as dioxygenin dCTP, biotin dcTP 7-azaguanosine, azidothymidine, inosine, or uridine.

In some embodiments, a biological sample may include a cell or a tissue sample and the biological sample may be subjected to in situ hybridization (ISH) using a probe. In some embodiments, a tissue sample may be subjected to in situ hybridization in addition to immunofluorescence (IF) to obtain desired information regarding the tissue sample.

Regardless of the type of probe and the target nucleic acid sequence, the specificity of binding between the probe and the nucleic acid sequence may also be affected depending on the binding conditions (for example, hybridization conditions in case of complementary nucleic acids). Suitable binding conditions may be realized by modulating one or more of pH, temperature, or salt concentration.

A probe may be intrinsically labeled (fluorophore attached during synthesis of probe) with a fluorophore or extrinsically labeled (fluorophore attached during a later step). For example, an intrinsically labeled nucleic acid may be synthesized using methods that incorporate fluorophore-labeled nucleotides directly into the growing nucleic acid chain. In some embodiments, a probe may be synthesized in a manner such that fluorophores may be incorporated at a later stage. For example, this latter labeling may be accomplished by chemical means by the introduction of active amino or thiol groups into nucleic acids chains. In some embodiments, a probe such a nucleic acid (for example, a DNA) may be directly chemically labeled using appropriate chemistries for the same.

In addition to probes for target nucleic acid sequences, nucleic acid sequence content may also be measured by DNA staining using ploidy markers. For example, Feulgen (or a fluorescent dye that binds double stranded DNA) staining may be performed to measure the amount of nucleic acid content within a nucleus. Although semiquatitative, Feulgen staining is capable of differentiating between cells with diploid chromosome from cells with hyperploidy (triploid, tetraploid cells, etc). As another example, fluorescently labeled centromeric probes may be used to quantitate chromosome content for individual chromosomes of interest. Classical Hodgkin lymphoma cells are generally hyperdiploid, so ploidy analysis may aid in the identification of RS/H cells as well as shed further light on their molecular changes.

### General Description of the Invention

The invention includes embodiments that relate generally to methods applicable in diagnostic or prognostic applications which enable multiple rounds of immunofluorescence detection on a single sample. The disclosed methods relate generally to detection, quantification and correlation of different target biomarkers from a single biological sample. In certain embodiments, the method enables multiple biomarkers to be detected on the same sample, thus correlations can be drawn among the multiple, different targets. The methods are particularly suited for the diagnosis of classical Hodgkin lymphoma, a neoplasm characterized by (1) the cancer cells are rare cells in a tissue sample, (2) generally only a small amount of biopsy sample is often available compared to most other cancers, (3) there is a need to test for multiple biomarkers for many patients. As currently established methods of diagnosing classical Hodgkin lymphoma all employ subjective methods for data normalization, the methods of the invention also provide objective measurements for a pathological diagnosis. It reduces the undiagnosed “grey zone” lymphomas and enables more effective individualized treatment for patients.

Detecting the targets in the same biological sample by the novel method may further provide relative, spatial information about the targets in the biological sample. Furthermore, the same detection channel may be employed for detection of different targets in the sample, enabling fewer chemistry requirements for analyses of multiple targets. The methods may further facilitate analyses based on detection methods that may be limited in the number of simultaneously detectable targets because of limitations of resolvable signals.

In some embodiments, the method of detecting multiple targets in a biological sample includes sequential detection of targets in the biological sample. The method generally includes the steps of detecting one or more first targets in the biological sample, optionally modifying the signal from the first targets, and detecting one or more second targets in the biological sample. The method may be repeated multiple rounds for detecting additional targets in the biological sample, and so forth.

In one embodiment, the invention provides a method for providing a composite image of a single biological sample which comprises: (1) generating a first series of images of the biological sample including the presence, absence and/or expression level of a first biomarker; (2) after signal removal from the first binder, generating one or more second series of images of the biological sample including the presence, absence and/or expression level of another biomarker; and (3) generating a composite image that provides the relative location or expression level of both the first biomarker and the other biomarker. In certain embodiments, step (2) is repeated for additional biomarkers until all biomarkers of interest are analyzed, and wherein each step (2) takes place after signal removal from the binders present from the previous step (2). In certain embodiments, the wavelength of the signals from binders used in step 1 and the different steps 2 may be the same—thus, the signals in each separate step 2 need not to be distinguishable.

The step of generating the first series of images of the biological sample comprises the steps of: (a) contacting the sample on a solid support with a first binder for a first biomarker; (b) staining the sample with a fluorescent marker that provides morphological information; (c) detecting, by fluorescence, signals from the first binder and the fluorescent marker; (d) generating the first images of at least part of the sample from the detected fluorescent signals.

In certain embodiments, step (d) comprises: (i) generating initial images of at least part of the sample from the detected fluorescent signals; and (ii) selecting a region of interest (ROI) from the initial images, and detecting by fluorescence, signals from at least the first binder and the fluorescent marker to generate the first images at a higher resolution than the initial images.

The first series of images include at least an image from the fluorescent signals from the first binder, an image from the fluorescent marker, and optionally an image that includes the fluorescent signals from both the first binder and the fluorescent marker. In certain other embodiments, the contacting steps include contacting the sample with a second binder for a second biomarker, and the second binder carries a fluorescent signal separately detectable from the other fluorescent signals. Thus, the first images may also include an image from the fluorescent signals from the second binder, and optionally an image containing the fluorescent signals from both the second binder and the fluorescent marker and/or an image containing the fluorescent signals from the first binder, the second binder and the fluorescent marker. The number of binders in the contacting step is only limited by practical concerns, e.g., the detection limit for different fluorescent markers, the spatial constrains/competition among the different binders and their corresponding targets. Thus, the contacting steps may include any number of additional binders for additional biomarkers, provided the binders each carry a fluorescent signal separately detectable from the other fluorescent signals, and the binding of one binder is not interfered by that of another. Thus, the first images may also include an image from each of the fluorescent signals from the additional binders and an image including signals from all the binders as well as the fluorescent marker.

In certain embodiments, the biological sample is from a patient suspected of having classical Hodgkin lymphoma. Thus, in a specific embodiment, the contacting step includes an antibody for, e.g., CD30, as the first binder, labeled with, e.g., Cy3. The fluorescent marker is DAPI. Optionally, the contacting step further includes an antibody for, e.g., CD 45, as the second binder, labeled with, e.g., Cy5. In another specific embodiment, the contacting step includes a primary antibody for, e.g., CD30, as the first binder, and a secondary antibody, e.g., labeled with Cy3 for signal detection.

Prior to the generation of the second series of images, the fluorescent signals from the binder(s) are removal (detailed below). The step of generating the second series of images of the biological sample comprises the steps of: (a) contacting the same sample with a binder for another biomarker; (b) optionally staining the sample with a fluorescent marker that provides morphological information; (c) detecting, by fluorescence, signals from the binder and the fluorescent marker; (d) generating the second images of at least part of the sample from the detected fluorescent signals. In certain embodiments, step (d) comprises: (i) obtaining the ROI information from step (1); and (ii) detecting by fluorescence, signals from at least the binder and the fluorescent marker to generate the second series of images at the same higher resolution as in step (1) above. In a preferred embodiment, the biomarker(s) detected in the step of generating the second series of images are different from the biomarker(s) detected in the first or previous.

Similar to the first series of images, the second series of images include at least an image from the fluorescent signal from the binder, an image from the fluorescent marker, and optionally an image that includes the fluorescent signals from both the binder and the fluorescent marker. In certain other embodiments, the contacting step includes contacting the sample with one or more binder(s) for one or more further biomarkers not detected elsewhere, and the binder(s) each carries a fluorescent signal separately detectable from the other fluorescent signals used. Thus, the second images may include an image from the fluorescent signals from the additional binder(s). The second images may also include respective images generated from the fluorescent signals from each further binder(s) and optionally one or more composite images comprising (i) respective images generated from signal from the each further binder(s) and the fluorescent marker; or (ii) an image generated from the signals from each binder and the fluorescent marker. The number of binders in the contacting step is only limited by practical concerns. Thus, the contacting steps may include any number of additional binders for additional target proteins, provided the binders each carry a fluorescent signal separately detectable from the other fluorescent signals, and the binding of one binder is not interfered by that of another. Thus, the second images may also include an image from each of the fluorescent signals from the additional binders and an image including signals from all the binders as well as the fluorescent marker.

In certain embodiments, the biological sample is from a patient suspected of having classical Hodgkin lymphoma. Thus, in a specific embodiment, the contacting step in generating the second series of images includes an antibody for one of the biomarkers other than the biomarker used in the step of generating the first series of images, e.g. CD30, as the second binder, labeled with, e.g., Cy3. The optional fluorescent marker is DAPI. Optionally, the contacting step further includes another antibody as an additional binder, labeled with, e.g., Cy5. In addition to CD30, biomarkers for classical Hodgkin lymphoma may include at least CD15, CD20, CD45, CD3, Pax-5; as well as CD79A, BOB1, OCT-2, etc. In a specific embodiment, the contacting step in generating the second series of images includes a primary antibody for, e.g., CD15, and a secondary antibody, e.g., labeled with Cy3, for signal detection.

The step of generating the second series of images of the biological sample may optionally be repeated for additional biomarkers until all biomarkers of interest are analyzed according to the embodiments of the invention.

In certain embodiments, the composite image in step (3) is generated by combining signal information from the higher resolution images for the ROI from the first and subsequent series of images. In other embodiments, the composite image is generated by a method comprising registering the location of signals from the fluorescent marker used in the higher resolution images. In still other embodiments, the generation of the composite image comprises registering the location of signals from the fluorescent marker acquired in step (1) with the location of signals from the fluorescent marker acquired in step (2).

In a specific embodiment, the method comprises generating a first low magnification image of a formalin fixed, paraffin embedded tissue sample from a patient suspected of having classical Hodgkin lymphoma, which has been stained by immunofluorescence for one or more biomarkers (e.g., CD30) and a fluorescent marker (e.g., DAPI); generating a virtual H&E or virtual DAB image from the low magnification image and using that to select regions of interest (ROI) based on the presence of the signal and staining intensity or morphology; generating a higher resolution image of the ROIs; removing the fluorescent signals; generating a second image at the same higher resolution, of the sample which has been stained by immunofluorescence for one or more other markers and optionally a fluorescent marker; overlaying or registering the images based on common images obtained using the fluorescent marker staining as co-ordinates to generate a composite image. Thus, in certain embodiments, the composite image is a brightfield type image, such as a virtual H&E or virtual DAB image. In certain embodiments, the method further comprises analyzing the images to measure for biomarker expression level in individual cells. In a preferred embodiment, the method comprises generating a first low magnification image of a tissue sample which has been stained by immunofluorescence for CD30; and using that to select regions of interest (ROI) for higher resolution imaging based on the presence of the signal and staining intensity or morphology.

In certain embodiments, instead of generating a composite image between the first series of images and the second series of images, the images are simply aligned and displayed side by side, for visual analysis by a pathologist. Thus, in certain embodiments, the method of aligning the images comprises a step of registering the location of signals from the fluorescent marker in the higher resolution, first images with the location of signals from the fluorescent marker in the second images.

In certain embodiments, the image analysis also includes an assessment of the tissue and/or cellular morphology of the biological sample.

In certain embodiments, the step of generating a second image is repeated with additional biomarkers of interest until all the biomarkers are analyzed. Thus, a composite image may be generated for any two or more biomarkers analyzed, such as all of the biomarkers analyzed.

In certain embodiments, in addition to the presence or absence of the biomarkers (binary assay) in the cancer cells of the biological sample, the relative level of expression is also measured for at least some of the biomarkers of interest. In certain embodiments, the expression level of a biomarker is measured from the signal intensity associated with the binder of the biomarker of interest. In certain embodiments, the expression level is compared to a reference level. The reference level may be pre-determined. In certain embodiments, the reference level may be determined using tissue with previously determined expression of the biomarker. Alternatively, signal intensity from adjacent cells (i.e., non-tumor cells) of the cells of interest (e.g., tumor cells) may be used as the reference level.

In certain embodiments, the inventive method is used to detect, in a patient sample suspected of suffering from classic Hodgkin lymphoma, the expression of at least CD30, CD15, CD45, PAX-5, and CD3.

In certain embodiments, the inventive method is used to detect, in a patient sample suspected of suffering from classic Hodgkin lymphoma, the expression of at least CD30, CD15, CD45, PAX-5, CD3, CD20, CD79A, BOB1 and OCT-2.

In certain embodiments, it is provided a method of analyzing a biological sample suspected of having classic Hodgkin lymphoma, comprising (A) detecting, in a single sample, for the expression of at least two biomarkers selected from CD30, CD15, CD20, CD45, CD3, Pax-5, CD79A, BOB1 and OCT-2; and (B) analyzing the sample based on the presence, absence and/or expression level of the at least two biomarkers. In certain embodiments, the expression of at least three of the biomarkers is analyzed. In certain other embodiments, the expression of at least four of the biomarkers is analyzed. In other embodiments, the expression of at least five of the biomarkers is analyzed. In certain embodiments, the expression of at least six of the biomarkers is analyzed. In certain other embodiments, the expression of at least seven of the biomarkers is analyzed. In other embodiments, the expression of at least eight of the biomarkers is analyzed. In a preferred embodiment, the expression of at least CD30, CD15, CD45, CD3, Pax-5 is analyzed. In another preferred embodiment, the expression of all the nine biomarkers is analyzed. In certain embodiments, the expression of at least the biomarkers CD30 and CD15 is analyzed.

In certain embodiments, the method of analyzing a biological sample suspected of having classic Hodgkin lymphoma, also comprised, in the detecting steps, detecting for expression of MUM1, fascin, EBV LMP-1, BCL-6, CD138, EMA, cytotoxic markers, ALK, CD43, kappa/lambda or pan T-cell markers.

In certain embodiments, the expression of the biomarkers is analyzed within individual cells.

In certain embodiments, the biological sample is a tissue sample.

In certain embodiments, the detection of each biomarker comprises contacting the sample with a binder that binds to said biomarker.

In other embodiments, it is provided a method for diagnosis of a classic Hodgkin lymphoma, comprising (1) analyzing a biological sample according certain embodiments of the invention; and (2) diagnosing whether the patient has classic Hodgkin lymphoma. In certain embodiments, the analyzing step also includes an assessment of the morphology of the sample.

In certain embodiments, the detecting step in the method of analyzing a biological sample suspected of having classic Hodgkin lymphoma, comprises (1) generating a first image of the biological sample, which step comprises: (a) contacting the sample on a solid support with a first binder for CD30; (b) detecting, by fluorescence, for the presence of signals from the first binder; and (c) generating the first image of at least part of the sample from the detected fluorescent signals; and (2) after signal removal from the first binder, generating a second image of the biological sample, which step comprises: (a) contacting the same sample with a binder for another biomarker; (b) detecting, by fluorescence, for the presence of signals from the binder; and (c) generating the second image of at least part of the sample from the detected fluorescent signals. In some embodiments, step (1)(c) comprises (i) generating an initial image of at least part of the sample from the detected fluorescent signals; and (ii) selecting a region of interest (ROI) from the initial image, and detecting by fluorescence, signals from the first binder to generate the first image at a higher resolution than the initial image. In some embodiments, step (2)(c) comprises (i) obtaining the ROI information from step (1); and (ii) detecting by fluorescence, signals from the binder for said another biomarker to generate the second image at the same higher resolution as in step (1). In a preferred embodiment, step (2) is repeated for additional biomarkers until all biomarkers of interest are analyzed, and wherein each step (2) takes place after signal removal from the binders present from the previous step (2).

In certain embodiments, the detecting step in the method of analyzing a biological sample suspected of having classic Hodgkin lymphoma, comprises

(1) generating a first series of images of the biological sample, which step comprises: (a) contacting the sample on a solid support with a first binder for a first of the biomarkers; (b) staining the sample with a fluorescent marker that provides morphological information; (c) detecting, by fluorescence, for the presence of signals from the first binder and the fluorescent marker; and (d) generating the first images of at least part of the sample from the detected fluorescent signals; and

(2) after signal removal from the first binder, generating one or more second series of images of the biological sample, which step comprises: (a) contacting the same sample with a binder for another of the biomarkers; (b) optionally staining the sample with a fluorescent marker that provides morphological information; (c) detecting, by fluorescence, for the presence of signals from the binder and the fluorescent marker; and (d) generating the second images of at least part of the sample from the detected fluorescent signals.

In certain embodiments, the detecting step (1)(d) comprises: (i) generating initial images of at least part of the sample from the detected fluorescent signals; and (ii) selecting a region of interest (ROI) from the initial images, and detecting by fluorescence, signals from at least the first binder and the fluorescent marker to generate the first images at a higher resolution than the initial images. In certain embodiments, the detecting step (2)(d) comprises: (i) obtaining the ROI information from step (1); and (ii) detecting by fluorescence, signals from at least the binder and the fluorescent marker to generate the second series of images at the same higher resolution as in step (1) above.

In certain embodiments, the first series of images include at least an image from the fluorescent signals from the first binder, an image from the fluorescent marker, and optionally an image that includes the fluorescent signals from the first binder and the fluorescent marker.

In certain embodiments, the contacting step (1)(a) includes contacting the sample with a second binder for a second of the biomarkers, and the second binder carries a fluorescent signal separately detectable from the other fluorescent signals in step (1); the first images include an image generated from the fluorescent signals from the second binder and optionally a composite image comprising (i) an image generated from signals from the second binder and the fluorescent marker and/or (ii) the first and second binder and the fluorescent marker.

In certain embodiments, the second series of images include at least an image from the fluorescent signals from the binder, an image from the fluorescent marker, and optionally an image that includes the fluorescent signals from the binder and the fluorescent marker.

In certain embodiments, the contacting step (2) (a) includes contacting the sample with one or more binder(s) for one or more further of the biomarkers not detected in step (1) and elsewhere in step (2), wherein the further binder(s) each carry a fluorescent signal separately detectable from the other fluorescent signals including from the other binder(s) used in the same step (2). In some embodiments, the second images include respective images generated from the fluorescent signals from each further binder(s) and optionally one or more composite images comprising (i) respective images generated from signal from the each further binder(s) and the fluorescent marker; or (ii) an image generated from the signals from each binder in step (2) and the fluorescent marker.

In certain embodiments, step (2) is repeated for additional biomarkers until all biomarkers of interest are analyzed, and wherein each step (2) takes place after signal removal from the binders present from the previous step (2).

In certain embodiments, the detecting step in generating the first series of images or the detecting step in generating the second series of images further comprises detecting autofluorescense of the biological sample.

In certain embodiments, the step of generating the first series of images further comprises: prior to generating the images of the sample, generating a lower resolution image of the entire solid support and locating the sample on the solid support.

In certain embodiments, generating the first images and/or generating the second images comprise generating brightfield type images that resemble a brightfield stain. In some embodiments, the brightfield type images resemble simulated H&E images. In other embodiments, the brightfield type images resemble simulated DAB images.

In certain embodiments, the detecting step in the method of analyzing a biological sample suspected of having classic Hodgkin lymphoma, further comprises an antigen retrieval step prior to the contacting step (1)(a).

In certain embodiments, the binders are antibodies specific for the target proteins. In some embodiments, the antibodies are labeled with a fluorophore.

In certain embodiments, the fluorescent marker is selected from the group consisting 4′,6-diamidino-2-phenylindole (DAPI), Eosin, Hoechst 33258 and Hoechst 33342 (two bisbenzimides), Propidium Iodide, Quinacrine, Fluorescein-phalloidin, Chromomycin A 3, Acriflavine-Feulgen reaction, Auramine O-Feulgen reaction or Ethidium Bromide. In some embodiments, the fluorescent marker stains the nucleus of a cell.

In certain embodiments, signal removal of the fluorescent signal is accomplished by a chemical agent. The chemical agent may be selected from the group consisting of sodium hydroxide, hydrogen peroxide, or sodium periodate.

In certain embodiments, the biological comprises a Formalin-Fixed, Paraffin-Embedded (FFPE) tissue sample, the first biomarker is CD30, and the fluorescent marker is DAPI. In certain preferred embodiments, the ROI selection is guided by signals from the binder for CD30.

In certain embodiments, the detecting step in the method of analyzing a biological sample suspected of having classic Hodgkin lymphoma, further comprising creating a RGB color blend heatmap image of the biomarker expression level by mapping the fluorescent signal from each of the binders for each of the biomarker to a reference color lookup table.

Thus, exemplary questions that may be answered for classical Hodgkin lymphoma, using the methods according to certain embodiments of the invention include:
definitive co-localization of CD30 and CD15: CD30 and CD15 are usually both positive in the RS/H cells of classical Hodgkin lymphoma, while other cells resembling the RS/H cells of classical Hodgkin lymphoma are usually CD30+, CD15— (e.g., normal cells in reactive lymph nodes, as well as cells in many cases of B- and T-cell lymphoma) or CD30−, CD15+(e.g., histiocytes);
co-localization of CD30 and CD45: CD30 and CD45 are usually CD30+, CD45− in the RS/H cells of classical Hodgkin lymphoma, while other cells resembling the RS/H cells of classical Hodgkin lymphoma may be CD30+, CD45+ (e.g., normal cells in reactive lymph nodes, as well as cells in many cases of B- and T-cell lymphoma), or CD30−, CD45+(e.g. normal cells in reactive lymph nodes, as well as many cases of B-cell lymphoma, including T-cell/histiocyte-rich B-cell lymphoma and nodular lymphocyte predominant Hodgkin lymphoma, and T-cell lymphoma);
B-cell program in Hodgkin lymphoma: CD30 vs. expression of CD20, PAX-5, CD79A, BOB1, OCT-2 (in normal lymph node tissue and most B-cell lymphomas, including T-cell/histiocyte-rich B-cell lymphoma and nodular lymphocyte predominant Hodgkin lymphoma, all five markers are typically expressed in most B-cells); for PAX-5, although in normal cells the staining is strong, in RS/H cells the staining is weak or moderate; most B cells express CD20, while in classical Hodgkin lymphoma cells, only about 20-25% cases express CD20, and the expression pattern is weaker and more inconsistent from cell to cell; the other three have about 10-15% incidence of expression on RS/H cells in different cases);

In addition to the above panel of biomarkers, additional biomarkers may be assayed to further improve the diagnosis of certain lymph node diseases. Thus, MUM1 may be added to help distinguish classical Hodgkin lymphoma from nodular lymphocytic predominance Hodgkin lymphoma; kappa/lambda may be added to identify B-cell lymphoma; and pan T-cell markers may be added for the differential diagnosis of T-cell lymphoma vs. classical Hodgkin lymphoma. Other biomarkers may also be assayed to further improve the diagnosis of certain lymph node diseases, for example fascin, EBV LMP-1, BCL-6, CD138, EMA, cytotoxic markers, ALK, CD43, etc.

In another embodiment, it is provided a method for diagnosis of a classic Hodgkin lymphoma, comprising detecting, in a single sample, the presence and expression level of CD30, CD15, CD20, CD45, CD3, Pax-5, CD79A, BOB1 and OCT-2; analyzing the presence and relative expression level of the biomarkers; and diagnosing whether the patient has classic Hodgkin lymphoma. The detecting step comprises:

(1) generating a first series of images of the biological sample, which step comprises: (a) contacting the sample on a solid support with an antibody for CD30; (b) staining the sample with DAPI; (c) detecting, by fluorescence, signals from a label of the antibody for CD30 and DAPI; and (d) generating the first images of at least part of the sample from the detected fluorescent signals;

(2) after signal removal, generating second series of images of the biological sample, which step comprises: (a) contacting the same sample with an antibody for CD15; (b) optionally staining the sample with DAPI; (c) detecting, by fluorescence, signals from a label of the antibody for CD15 and DAPI; and (d) generating the second images of at least part of the sample from the detected fluorescent signals; and

(3) repeat step (2) for at least four more rounds, with differentially labeled antibody specific for CD20 and Pax-5, CD45 and CD3, CD79a and Oct2, and labeled antibody for BOB1, respectively.

In still another embodiment, it is provided a method for diagnosis of a classic Hodgkin lymphoma, comprising detecting, in a single sample, the presence and expression level of CD30, CD15, CD20, CD45, CD3, Pax-5, CD79A, BOB1 and OCT-2; analyzing the presence and relative expression level of the biomarkers; and diagnosing whether the patient has classic Hodgkin lymphoma. The detecting step comprises:

(1) generating a first series of images of the biological sample, which step comprises: (a) contacting the sample on a solid support with an antibody for CD30 and labeled antibody specific for BOB1; (b) staining the sample with DAPI; (c) detecting, by fluorescence, signals from a label of the antibody for CD30, the labeled antibody specific for BOB land DAPI, wherein the signals are distinguishable from each other; and (d) generating the first images of at least part of the sample from the detected fluorescent signals;

(2) after signal removal, generating second series of images of the biological sample, which step comprises: (a) contacting the same sample with an antibody for CD15 and labeled antibody specific for Oct2; (b) optionally staining the sample with DAPI; (c) detecting, by fluorescence, signals from a label of the antibody for CD15, the labeled antibody specific for Oct2 and DAPI, wherein the signals are distinguishable from each other; and (d) generating the second images of at least part of the sample from the detected fluorescent signals; and

(3) repeat step (2) for at least three more rounds, with differentially labeled antibody specific for CD20 and Pax-5, CD45 and CD3, and labeled antibody for CD79a, respectively.

The invention includes embodiments that enable detection of both protein biomarkers and other biomarkers, such as polysaccharide (e.g., CD 15) or nucleic acid sequences of interest or nucleic acid or chromosome content. Thus, in addition to multiple rounds of immunofluorescence detection on the single sample, the sample may be further analyzed for the presence and amount of specific nucleic acid sequences using methods such as fluorescence in situ hybridization (FISH). The sample may be further analyzed for the amount of nucleic acid or chromosome content in a single cell, by measuring the amount of nucleic acid content using fluorescent or non-fluorescent dyes that bind double stranded DNA, or using nucleic acid probes that bind chromosomal DNA non-specifically or that binds the centromere. Images obtained in these analyses may be combined with images from the protein biomarker assay steps to generate the composite image for the diagnosis of classical Hodgkin lymphoma.

### Generating an Image of the Biological Sample

In certain embodiments of the invention, the method comprises a step of generating a first series of images of the biological sample from a patient suspected of having classical Hodgkin lymphoma. The first series of images are generated by (a) contacting the sample on a solid support with a first binder for a first biomarker; (b) staining the sample with a fluorescent marker that provides morphological information; (c) detecting, by fluorescence, signals from the first binder and the fluorescent marker; and (d) generating the first images of at least part of the sample from the detected fluorescent signals.

In certain embodiments of the invention, the method further comprises a step of generating a second series of images of the biological sample from the patient suspected of having classical Hodgkin lymphoma. The second series of images are generated by, after removal of fluorescent signal for the first series of images, (a) contacting the same sample with a binder for another biomarker; (b) optionally staining the sample with a fluorescent marker that provides morphological information; (c) detecting, by fluorescence, signals from the binder and the fluorescent marker; and (d) generating the second images of at least part of the sample from the detected fluorescent signals.

In certain embodiments, step (c) of both steps also comprises detecting, by fluorescence, an endogenous fluorescence signal (also known as autofluorescence) originating from such structures as red blood cells, fibroses, and lipofuscin granules.

In certain embodiments, generating the first series of images (step (1)(d)) comprises, generating initial images of at least part of the sample from the detected fluorescent signals; selecting a region of interest from the initial images, and detecting by fluorescence, signals from at least the first binder and the fluorescent marker to generate the first series of images at a higher resolution than the initial images. By “selecting a region of interest”, it is understood to mean (1) a user selects a region of interest based on the initial images; (2) the computer (i.e., imaging system implementing the method) selects a region of interest based on the initial images, an algorithm, and an instruction it received; or (3) the computer selects a region of interest based on the initial images and an algorithm. It is to be understood that the first images do not necessarily refer to the initial images generated. Similarly, the second images do not literally refer to the very second images generated by the embodiments of the method.

Thus, in certain embodiments, generating the second series of images (step (2)(d)) comprises, obtaining the ROI information from step (1); and detecting by fluorescence, signals from at least the binder and the fluorescent marker to generate the second series of images at the same higher resolution as in step (1) above.

In certain embodiments, signals from the fluorescent marker are acquired in order to allow images to be registered.

In certain embodiments of the invention, the step of generating a second series of images of the biological sample is cycled until all of the biomarkers are analyzed according to embodiments of the invention.

In certain embodiments, the images obtained may be one or more brightfield type images that resemble a brightfield staining protocol. Thus, the fluorescence image data may be used to generate a simulated (virtual) hematoxylin and eosin (H&E) image via an algorithm. Alternatively, or in addition, a simulated (virtual) 3,3′-Diaminobenzidine (DAB) image may be generated via a similar algorithm. Detailed methods for converting fluorescence image data into a brightfield type image is described hereinbelow under the heading “Image Acquisition and Analysis”. The brightfield type image is used for the selection of the region of interest, as well as the analysis of the biological sample and the diagnosis of classical Hodgkin lymphoma.

In some embodiments, a biological sample may include a tissue sample. In some embodiments, the tissue sample may be first fixed and then dehydrated through an ascending series of alcohols, infiltrated and embedded with paraffin or other sectioning media so that the tissue sample may be sectioned. In an alternative embodiment, a tissue sample may be sectioned and subsequently fixed. In some embodiments, the tissue sample may be embedded and processed in paraffin. Examples of paraffin that may be used include, but are not limited to, Paraplast, Broloid, and Tissuecan. Once the tissue sample is embedded, the sample may be sectioned by a microtome into sections that may have a thickness in a range of from about three microns to about five microns. Once sectioned, the sections may be attached to slides using adhesives. Examples of slide adhesives may include, but are not limited to, silane, gelatin, poly-L-lysine. In certain embodiments, if paraffin is used as the embedding material, the tissue sections may be deparaffinized and rehydrated in water. The tissue sections may be deparaffinized, for example, by using organic agents (such as, xylenes or gradually descending series of alcohols).

In some embodiments, aside from the sample preparation procedures discussed above, the tissue section may be subjected to further treatment prior to, during, or following immunofluorescence assay. For example, in some embodiments, the tissue section may be subjected to epitope (i.e., antigen) retrieval methods, such as, heating of the tissue sample in citrate buffer. In some embodiments, a tissue section may be optionally subjected to a blocking step to minimize any non-specific binding.

Following the preparation of the sample, the sample may be contacted with a binder solution (e.g., labeled-antibody solution in an immunofluorescence procedure) for a sufficient period of time and under conditions suitable for binding of binder to the biomarker (e.g., antigen in an immunofluorescence procedure). In some embodiments, the biological sample may be contacted with more than one binder in the contacting step during the step of generating the first or the second images. The plurality of binders may be capable of binding different biomarkers in the biological sample. For example, a biological sample may include two target proteins: CD30 and CD45 and two sets of binders may be used in this instance: anti-CD30 (capable of binding to CD30) and anti-CD45 (capable of binding to CD45). A plurality of binders may be contacted with the biological sample simultaneously (for example, as a single mixture).

In addition to contacting the sample with one or more binders for one or more targets, the sample may also be stained with at least one additional binder that provides morphological information. In one embodiment, the binders that provide morphological information may be included simultaneously with the binders for the targets. In other embodiments, they may be used to stain the sample after the binder-target reaction.

The morphological information includes, but is not limited to, tissue morphology information such as tissue type and origin, information about the origin of certain cells, information about subcellular structure of cells such an membrane, cytoplasm, nucleolar or nucleus, information about cell differentiation state, cell cycle stage, cell metabolic status, cell necrosis or apoptosis, cell types, and tumor, normal, and stromal regions. For example, the morphological information may comprise information about cytoplasmic localization of cells of epithelial origin or it may indicate localization of a poorly differentiated or necrotic region of a tumor.

In some embodiments the at least one additional binder for morphological targets is an antibody that binds to, but is not limited to, the following target protein:
Cytokeratin: marker for epithelial cells
Pan-cadherin: marker for the cell membrane
Na+K+ATPase marker for cell membrane
Smooth muscle actin: smooth muscle cells, myofibroblasts and myoepithelial cells
CD31, CD34 marker for blood vessels
Ribosomal protein S6: marker for cytoplasm
Glut 1 marker for hypoxia
Ki67 marker for proliferating cells
Collagen IV stroma
marker for nucleolar
ASH2L marker for nucleolar
eIF6 marker for nucleolar

Other targets that provide morphological information may also include keratin 15, 19, E-cadherin, Claudin 1, EPCAM, fibronectin and vimentin.

The endogenous fluorescence (autofluorescence) of tissue may be used to provide additional morphological information including, but not limited to, red blood cells, lipofuscin granules, and fibrosis in the sample under study.

Preferably, the binders are labeled with fluorophores. When more than one target are detected, the binders for each target are preferably labeled with different fluorophores which have different emission wavelengths such that the signals can be independently detected and do not overlap substantially. Also preferably, the optional binders that provide morphological information are also labeled with different fluorophores from the other binders such that they have different emission wavelengths as well.

After a sufficient time has been provided for the binding action, the sample may be contacted with a wash solution (for example an appropriate buffer solution) to wash away any unbound probes. Depending on the concentration and type of probes used, a biological sample may be subjected to a number of washing steps with the same or different washing solutions being employed in each step.

Following the reaction between the binders and the target biomarkers, the sample is further stained with a fluorescent marker that provides additional morphological information. The term “fluorescent marker” refers to a fluorophore which selectively stains particular parts of a tissue or other biological sample, such as certain subcellular morphology. Examples of suitable fluorescent marker (and their target cells, subcellular compartments, or cellular components if applicable) may include, but are not limited to: 4′,6-diamidino-2-phenylindole (DAPI) (nucleic acids), Eosin (alkaline cellular components, cytoplasm), Hoechst 33258 and Hoechst 33342 (two bisbenzimides) (nucleic acids), Propidium Iodide (nucleic acids), Quinacrine (nucleic acids), Fluorescein-phalloidin (actin fibers), Chromomycin A 3 (nucleic acids), Acriflavine-Feulgen reaction (nucleic acid), Auramine O-Feulgen reaction (nucleic acids), Ethidium Bromide (nucleic acids). Nissl stains (neurons), high affinity DNA fluorophores such as POPO, BOBO, YOYO and TOTO and others, and Green Fluorescent Protein fused to DNA binding protein (e.g., histones), ACMA, and Acridine Orange. Preferably, the fluorescent marker stains the nucleus. More preferably, the fluorescent marker comprises 4′,6-diamidino-2-phenylindole (DAPI).

The total number of binders and fluorescent marker that may be applied to a biological sample in each round of image generation may depend on the spectral resolution achievable by the spectrally resolvable fluorescent signals from the fluorophores used. Spectrally resolvable, in reference to a plurality of fluorophores, implies that the fluorescent emission bands of the fluorophores are sufficiently distinct, that is, sufficiently non-overlapping, such that, the respective fluorophores may be distinguished on the basis of the fluorescent signal generated by each fluorophore using standard photodetection systems. In some embodiments, a biological sample may be reacted with ten or less than ten fluorophores in each round of detection by a detection system. In other embodiments, a biological sample may be reacted with six or less than six fluorophores in each round of detection by a detection system.

Signals from the binder-labeled fluorophores, the fluorescent marker, and the autofluorescence of the sample may be detected using a detection system. The detection system may include a fluorescent detection system. In some embodiments, signal intensity, signal wavelength, signal location, signal frequency, or signal shift may be determined. In some embodiments, one or more aforementioned characteristics of the signal may be observed, measured, and recorded. In some embodiments, fluorescence wavelength or fluorescent intensity may be determined using a fluorescent detection system. In some embodiments, a signal may be observed in situ, that is, a signal may be observed directly from the fluorophore associated through the binder to the target in the biological sample.

In some embodiments, observing a signal may include capturing an image of the biological sample. In some embodiments, a microscope connected to an imaging device may be used as a detection system, in accordance with the methods disclosed herein. In some embodiments, a fluorophore may be excited and the fluorescent signal obtained may be observed and recorded in the form of a digital signal (for example, a digitalized image). The same procedure may be repeated for different fluorophores that are bound in the sample, and for the autofluorescence of the sample, using the appropriate fluorescence filters.

Additional details about the method and system for fluorescence detection, as well as the method and system for generating a image of the sample are provided hereinbelow under the heading “Image Acquisition and Analysis”.

In some embodiments, after the first series of images of the biological sample is generated from the detected fluorescent signals, and prior to the generation of the second series of images, the fluorescent signals from the binders are modified. A chemical agent may be applied to the biological sample to modify the fluorescent signal. In some embodiments, signal modification may include one or more of a change in signal characteristic, for example, a decrease in intensity of signal, a shift in the signal peak, a change in the resonant frequency, or cleavage (removal) of the signal generator resulting in signal removal. Such chemical agents are known to person skilled in the art, for example, see U.S. Pat. No. 7,629,125.

In some embodiments, a chemical agent may be in the form of a solution and the biological sample may be contacted with the chemical agent solution for a predetermined amount of time. The concentration of the chemical agent solution and the contact time may be dependent on the type of signal modification desired. In some embodiments, the contacting conditions for the chemical agent may be selected such that the binder, the target, the biological sample, and binding between the binder and the target may not be affected. In some embodiments, a chemical agent may only affect the fluorophore and the chemical agent may not affect the target/binder binding or the binder integrity. Thus by way of example, a binder may include a primary antibody or a primary antibody/secondary antibody combination. A chemical agent may only affect the fluorophore, and the primary antibody or primary antibody/secondary antibody combination may essentially remain unaffected. In some embodiments, a binder (such as, a primary antibody or primary antibody/secondary antibody combination) may remain bound to the target in the biological sample after contacting the sample with the chemical agent. In some embodiments, a binder may remain bound to the target in the biological sample after contacting the sample with the chemical agent and the binder integrity may remain essentially unaffected (for example, an antibody may not substantially denature or elute in the presence of a chemical agent).

In some embodiments, a characteristic of the signal may be observed after contacting the sample with a chemical agent to determine the effectiveness of the signal modification. For example, fluorescence intensity from a fluorescent signal generator may be observed before contacting with the chemical agent and after contacting with the chemical agent. In some embodiments, a decrease in signal intensity by a predetermined amount may be referred to as signal modification. In some embodiments, modification of the signal may refer to a decrease in the signal intensity by an amount in a range of greater than about 50 percent. In some embodiments, modification of the signal may refer to a decrease in the signal intensity by an amount in a range of greater than about 60 percent. In some embodiments, modification of the signal may refer to a decrease in signal intensity by an amount in a range of greater than about 80 percent. In certain embodiments, the signal modification may be accomplished through oxidation, stripping, photobleaching, or a mixture thereof. In a preferred embodiment, the chemical agent is selected from the group consisting of sodium hydroxide, hydrogen peroxide, or sodium periodate. In another embodiment signal modification may be accomplished by contacting the sample with light and/or chemical agent, as described more fully in U.S. patent application Ser. No. 13/336,409 entitled “PHOTOACTIVATED CHEMICAL BLEACHING OF DYES FOR USE IN SEQUENTIAL ANALYSIS OF BIOLOGICAL SAMPLES” and filed on Dec. 23, 2011, herein incorporated by reference in its entirety.

In some embodiments, signal modification/removal is performed after each round of image generation, before the sample is contacted with fluorescently-labeled binders for additional and different targets for the generation of the next series of images.

In certain embodiments of the invention, the method further comprises a step of analyzing a nucleic acid sequence of interest, or nucleic acid or chromosome content of the biological sample. These analyses are performed after the target protein or other non-nucleic acid sequence biomarker images have been acquired. Here, the following steps may be performed: (a) contacting the sample with a probe for each of at least one target nucleic acid sequences thus hybridizing the probes with the nucleic acid sequences; or, alternatively, contact the sample with a dye which binds nucleic acid sequence; (b) optionally, staining the sample with the fluorescent marker used in generating the target protein or other non-nucleic acid sequence biomarker images; (c) detecting, by fluorescence, signals from the probes for each of the target nucleic acid sequences or the dye, and the fluorescent marker; (d) generating images of the sample from the detected fluorescent signal or signals; and (e) analyze the nucleic acid sequence of interest or the nucleic acid/chromosome content.

In certain embodiments, prior to the nucleic acid based analysis step, the method further comprises digesting the sample by a protease. The breaking of peptide bindings by protease digestion directly affects signal quality as it eases access of the probes/dyes to the target nucleic acid and reduces autofluorescence generated by intact proteins. Protease digestion also serves to remove the binder from the target protein(s) and therefore removes the immunofluorescence signal associated with the binders. An exemplary protease for a protease digest is a serine protease such as proteinase K. Another exemplary protease is a carboxyl protease, such as pepsin.

Preferably, the probes are fluorescently labeled.

Methods for the detection of nucleic acid sequence such as hybridization are well known. In certain embodiments, a specific nucleic acid sequence is detected by FISH (or a variation of FISH such as IQ-FISH), polymerase chain reaction (PCR) (or a variation of PCR such as in-situ PCR), RCA (rolling circle amplification) or PRINS (primed in situ labeling). In an exemplary embodiment, the specific nucleic acid sequence is detected by FISH. Thus, the target nucleic acid sequence in the biological sample is denatured and hybridizes, in situ, with a denatured fluorescently labeled probe. In certain preferred embodiments, when the target nucleic acid sequence is analyzed by FISH, a chromosome specific probe, such as a centromere probe for the same chromosome, is used, together with the probe for the target nucleic acid sequence. The signal from a chromosome specific probe shows whether the target nucleic acid sequence is on the same chromosome. Preferably, the chromosome specific probes are labeled with a fluorophore which generate a signal distinct from that of the probe for the target nucleic acid sequence.

Following the hybridization reaction, the sample is optionally stained with the fluorescent marker which provides additional morphological information. The fluorescent marker preferably is the same as used for obtaining the target protein or other non-nucleic acid sequence biomarker images. Alternatively, the fluorescent marker is different but stains the same subcellular compartment as that used for obtaining the first image. In certain embodiments, fluorescent signal from the staining for the first image is sufficiently retained so this step is optional. In other embodiments, the fluorescent signal from the staining for the first image has faded and the sample is stained as provided here.

In certain embodiments, it is preferred that the fluorescent marker stains the nucleus of the cell. Thus the staining assists the focusing of the FISH signal. By obtaining the focused nucleus, the FISH signals can be captured by imaging several focal planes above and below the focused nucleus. The staining also assists the counting of the FISH signals. Since FISH signals may be scattered throughout the nucleus, dot counting performed using a single focal plane may lead to missed counts. However, by capturing several z-stacks within each field of view, it provides more data to generate, as close as possible, a three dimensional view of the nucleus. Therefore it is provided a more accurate method of counting FISH signals.

Staining the biological sample with the same fluorescent marker or fluorescent markers that stain the same subcellular compartment also serves to provide reference points for aligning or overlaying the first image and the second image. Thus, it facilitates the proper alignment of the images for review. It also facilitates the generation of a composite image. For details on the overlay of the first and second image, see the section “Image Acquisition and Analysis” below.

Signals from the probe-labeled fluorophores and the fluorescent marker may be detected using a detection system as discussed above. Additional details about the method and system for fluorescent detection are provided hereinbelow under the heading “Image Acquisition and Analysis”.

In some embodiments, after the image of the biological sample is generated from the fluorescent signals of the probes/dye, the fluorescent signals from the probes/dye for each of the target nucleic acid sequences are modified by, for example, oxidation, stripping, photobleaching, or a mixture thereof. Thereafter, one or more additional images are obtained following the method herein described before. Namely, each additional image is generated by (1) contacting the sample with a probe for each of at least one additional target nucleic acid sequences thus hybridizing the probes with the sequences; (2) optionally, staining the sample with the fluorescent marker; (3) detecting, by fluorescence, signals from the probes for each of the additional sequences and the fluorescent marker; and (4) generating an image of the sample from the detected fluorescent signal.

### Image Acquisition and Analysis

In certain embodiments, both the first series of images and the second series of images of the entire biological sample are obtained at high resolution. Thus, the emission from each fluorophore is measured at its emission wavelength at high resolution. By high resolution, it is meant that the images were obtained at a resolution between 20× to 100×, corresponding to a numerical aperture between 0.5 and 1.4, supporting a pixel size of 75-375 nm. Preferably, the images are obtained at 40×, at a numerical aperture of about 0.85 and pixel size of about 170 nm. Image capture at 40× is preferable since the resolution is high enough to capture the binder signals while capturing relatively large field of views compared to a 60× or 100×.

In other embodiments, the biological sample may not occupy the entire surface of the solid support, or a high resolution image of the entire biological sample may not be necessary. Thus, while obtaining the first image, the entire surface of the solid support may be first scanned at a low resolution such as at 2× or 1.25×. An image analysis algorithm is then applied to the low resolution image and detects the area that contains the biological sample. Coordinates that mark the border of the biological sample are captured and used to direct subsequent higher resolution scan(s). The measurement of emission from one of the fluorophores may be sufficient to obtain the coordinates for the border of the sample.

Thus, the area that contains the biological sample may be detected by a computer implemented method comprising: obtaining an image of the biological sample using at least one processor; segmenting the image with the processor into a plurality of regions using either (a) a maximum a posteriori marginal probability (MPM) process with a Markov Random Field (MRF), or (b) a maximum a posteriori (MAP) estimation with a Markov Random Field (MRF); and classifying the plurality of regions into a background region and a tissue region to form a binary mask. The method may also comprise applying an active contour method to the binary mask to refine the biological sample boundary.

In still other embodiments, a higher resolution image of the entire biological sample may not be necessary. Rather, a higher resolution image is only required for selected regions of interest (ROI) of the sample. Thus, while generating the first image, the biological sample is first imaged at a lower resolution (such as at 10×, compared to the higher resolution) which enables ROI selection. Optionally, imaging at lower resolution includes a scan for each of the fluorophores used in the contacting and staining step. One or more ROIs may be selected based on predefined criteria (e.g., sample integrity, phenotype such as tumor or normal, muscle or duct tissue etc.). In certain embodiments, the ROIs are selected based, at least in part, on expression level of the target biomarker(s) detected from the first binder. Thus, certain ROIs may be selected for a lower target biomarker expression level compared to a first threshold, while other ROIs may be selected due to a higher biomarker expression level compared to a second threshold (which may be different from the first threshold). The coordinates of the ROIs are used to direct the higher resolution scanning to the ROIs only. In certain embodiments, the second image is obtained for the ROIs alone, at the same higher resolution as the image obtained for the ROIs for the first image. As an example, about 5 to 20ROIs may be selected for a sample suspected of having classical Hodgkin lymphoma. In a preferred embodiment, the criteria for ROI selection may be a positive signal for CD30. In another preferred embodiment, the criteria for ROI selection may be a positive signal for CD30 in large, RS or RS/H cells.

In certain embodiments, the method provides a composite image of a single biological sample and includes generating a first series of images of the biological sample and generating a second series of images of the biological sample. These first and second series of images are generated by (1) fluorescent detection of the signals from the biological sample and the fluorescent marker that provides morphological information, and (2) generating the first and second series of images of the sample from the detected fluorescent signals, respectively. These steps are preferably performed using a fluorescence microscope and repeated for each of the fluorophores used. Thus, each fluorophore is excited and its fluorescent emission measured at its wavelength using a standard instrument such as a CCD camera or a fluorescent scanner. Optionally, autofluorescence of the biological sample is also measured and its effect on the measurement of certain fluorophore is taken into consideration. For example, an algorithm may be used to subtract out background autofluorescence at one or more emission wavelengths.

In certain embodiments, a composite image is generated that provides the relative location and expression of both the first target biomarker and the other target biomarker. In certain embodiments, the composite image is dynamically generated. Thus, a composite image may be dynamically generated by combining any two or more images from the first and the second series of images. Further, more than one composite image may be generated based on different combinations of the first series of images and the second series of images. Thus, in certain embodiments, the composite image is generated by combining signal information from the higher resolution images from the first series of images and the second images of the same resolution. In some embodiments, the method comprises a step of registering the location of signals from the fluorescent marker in the higher resolution, first images with the location of signals from the fluorescent marker in the second images.

In certain embodiments, instead of generating a composite image among the first series of images and the second series of images, the images are simply aligned and displayed side by side, for visual analysis by a pathologist. Thus, in certain embodiments, the method of aligning the images comprises a step of registering the location of signals from the fluorescent marker in the higher resolution, first images with the location of signals from the fluorescent marker in the second images.

In some embodiments, the first series of images include at least an image from the fluorescent signals from the first binder, an image from the fluorescent marker, and optionally an image that includes the fluorescent signals from the first binder and the fluorescent marker.

In some embodiments, the second series of images include at least an image from the fluorescent signals from the binder, an image from the fluorescent marker, and optionally an image that includes the fluorescent signals from the second binder and the fluorescent marker.

As described above, in certain embodiments, the initial image (i.e., lower resolution image) is first converted into one or more brightfield type image that resemble a brightfield staining protocol. Thus, the initial fluorescence image data may be used to generate a simulated (virtual) hematoxylin and eosin (H&E) image via an algorithm. Alternatively, a simulated (virtual) 3,3′-Diaminobenzidine (DAB) image may be generated via a similar algorithm. Detailed methods for converting fluorescence image data into a brightfield type image is described hereinbelow. The brightfield type image is then used for the selection of the region of interest, based at least in part, on target biomarker expression, and optionally on morphological information.

In certain embodiments, the image of the entire biological sample or selected ROIs within the sample may not be obtainable with a single scan due to the limitation of the microscope's field of view (FOV). That is, the area to be imaged may be larger than the microscope FOV can capture. In such cases the desired image may be acquired by capturing multiple FOVs across the slide or selected ROI. These raw images of the FOVs are corrected to adjust for field variation and may be then stitched together according to an algorithm that aligns the separate FOVs into a single image of the entire slide or ROI. Such image stitching algorithms are well-known to a person skilled in the art, see U.S. Pat. No. 6,674,884. Monochrome cameras are often used in fluorescent imaging because of their higher sensitivity and ability to capture predetermined wavelengths by utilizing the appropriate excitation and emission filters along with dichroic minor. Thus, gray scale images for individual channels are generated. The gray scale digital images for each fluorescent channels may be pseudo-colored and merged to populate the desired image.

In a preferred embodiment, generating the first series of images comprises (1) optionally generating a lower resolution image of the entire solid support and locating the sample on the solid support; (2) generate a medium resolution image of the sample; (3) identify regions of interest (ROI) according to predetermined criteria; and generating a higher resolution image for each of the ROIs. The second series of images generated is a higher resolution image of each of the ROIs selected during the generation of the first series of image. In these embodiments, the term lower, medium and higher is not limited to certain magnifications. Rather, they are relative to each other. In a most preferred embodiment, the low resolution image is a 2× image; the medium resolution images are 10× images and the high resolution images are 40× images. In certain embodiments, it may be desirable to enhance the images by computer-aided means to more clearly illustrate the characteristics of the target biomarkers. Thus, one example creates a RGB color blend heatmap image where target expression levels are mapped to a reference color lookup table. An example of this lookup table would map low level intensities to shades of blue, intermediate intensities to shades of yellow and high intensities to shades of red for easier identification of areas with different levels of staining intensity. In another example, a color blended composite image is created for each of the first series of images, to better display the spatial relationship among the target biomarker and the fluorescent marker. In still another example, a pseudo-color image of a particular fluorophore channel may be created. For example signals for CD30 would be colored red and signals for CD15 would be colored green making it easy to distinguish relative amounts of the two types of signals in a given cell or area of tissue.

In certain embodiments, the first and second series of images are aligned, preferably according to, at least in part, some of the images obtained from the signals detected from the fluorescent marker. In certain embodiments, the first and second images are overlaid and a composite image is further created. A composite image allows direct comparison of results obtained from the first image with that from the second image on a cell by cell basis.

A composite image may not include the whole image of the first or the second image, or all of the signals acquired in the generation of the first image or the second image. The images obtained from the fluorescent marker may contain any morphological information, and may include images of a particular subcellular component from the biological sample, such as the cell nucleus. Thus, an algorithm acquires coordinates from the morphological information (e.g., subcellular components) in the first and second image, and uses these to align the first and the second image. In a preferred embodiment, the morphological information used for the alignment of the image is at the cell level. In a more preferred embodiment, the morphological information used for the alignment of the image is at the subcellular level. In a most preferred embodiment, the morphological information used for the alignment of the images is derived from the fluorescent signal of cell nuclei.

A composite image may not include the whole of the first or the second images, or all of the signals acquired in the generation of the first images or the second images. Because of shifts in the position of the slide and the microscope stage, the second images may be rotated or translated with respect to the first image, and this rotation or translation must be corrected for aligning or registering the two images prior to producing a composite image.

To register the images, it is preferred to use an identical morphological marker in the first image and the second image. An example of such a marker is DAPI. The images obtained from the fluorescent marker provide morphological information regarding particular subcellular compartments in both images, and the relative location of said subcellular compartments remains substantially unchanged in the two images. Thus, an algorithm can use this spatial information to establish a coordinate transformation between the first images and the second images by (a) calculating the Fourier transformations of the images; (b) transforming the amplitude components Fourier transformations into log-polar co-ordinates, creating a translation-invariant signature of each of the images; (c) applying a second Fourier transform to the signatures; (d) calculating the correlation function between the signatures; (e) inverse Fourier transforming the correlation function, solving for rotation and scaling between the images; (f) applying the rotation and scale to the second images so that the images are rotated and scaled identically; (g) calculating the cross-power correlation function between the identically-scaled images; and (h) inverse Fourier transforming the cross power correlation, yielding the translation between the first images and the second images. The translation, rotation and scale are then used to produce identically-aligned (registered) images. The cross-power correlation is preferred to the conventional product-moment correlation because it is insensitive to intensity differences between the two images and to slowly-varying intensity differences across the field of view of the microscope.

In certain embodiments, the first and the second images, as well as any composite images created are utilized to characterize the expression of the target biomarkers. Thus, the biomarkers expression level may be analyzed by correlating an intensity value of a signal (for example, fluorescence intensity) to the amount of target in the biological sample. A correlation between the amount of target and the signal intensity may be determined using calibration standards. In some embodiments, one or more control samples may be used. By observing the presence or absence of a signal in the samples (biological sample of interest versus a control), information regarding the biological sample may be obtained. For example by comparing a diseased tissue sample versus a normal tissue sample, information regarding the targets present in the diseased tissue sample may be obtained. Similarly by comparing signal intensities between the samples (i.e., sample of interest and one or more control), information regarding the expression of targets in the sample may be obtained.

The methods disclosed herein may find applications in analytic, diagnostic, and therapeutic applications in biology and in medicine. Analysis of cell or tissue samples from a patient, according to the methods described herein, may be employed diagnostically (e.g., to identify patients who have a classical Hodgkin lymphoma), or prognostically (e.g., to identify patients who are likely to develop a particular disease, respond well to a particular therapeutic or be accepting of a particular organ transplant). The methods disclosed herein may facilitate accurate and reliable analysis of a plurality of targets (e.g., disease markers) from the same biological sample.

In certain embodiments, the first and/or the second and/or the additional fluorescent images are converted into brightfield type images that resemble a brightfield staining protocol. Thus, the fluorescence signal detected from the fluorescent marker, and any autofluorecence of the biological sample may be used to generate a simulated (virtual) hematoxylin and eosin (H&E) image via an algorithm. Alternatively, a simulated (virtual) 3,3′-Diaminobenzidine (DAB) image may be generated via a similar algorithm. In certain embodiments, the virtual H&E image includes signals from the fluorescent marker (e.g., DAPI), and autofluorescence. In certain embodiments, the virtual DAB image includes signals from the fluorescent marker and the binder for the target biomarker (i.e., anti-CD30 antibody).

Methods for converting fluorescent images into a pseudo brightfield image are known. Also known is a method that creates a brightfield image from fluorescent images wherein structural features and details of the biological sample are identified as if the image was obtained directly from a specified brightfield staining protocol. U.S. Pat. No. 8,269,827. In certain embodiments of the current invention, an improved method for generating a brightfield type image that resembles a brightfield staining protocol of a biological sample is used, as described more fully in K. Kenny, U.S. patent application Ser. No. 13/211,725 entitled “SYSTEM AND METHODS FOR GENERATING A BRIGHTFIELD IMAGE USING FLUORESCENT IMAGES” and filed on Aug. 17, 2011, herein incorporated by reference in its entirety. The method involves the use of a calibration function obtained from a brightfield image of a biological sample or defined using a preselected or desired color. The preselected or desired color may be chosen by an operator, which may be a pathologist or microscopist familiar with standard biological staining protocols. The calibration function estimates an intensity transformation that maps the fluorescent images into the brightfield color space using three parameters, a[Red], a[Green], a[Blue], called the “extinction coefficients.”.

The estimated parameters may be derived by preparing one or more biological specimens with a wide range of staining intensity in the biomarker of interest, labeled with a visible dye such as hematoxylin, eosin, or diaminobenzidine (DAB). The sample may then be imaged in brightfield, and the distribution of red, green, and blue pixel intensity levels may be calculated; the pixel intensity levels are normalized to the interval [0,1]. The color with the smallest value for mean(log intensity) is identified. Without loss of generality, one may presume a specific color. For example, if the color is green, the mean values of (log Red/log Green) and (log Blue/log Green) are calculated, and the triple, (mean[log Red/log Green], 1, mean[log Blue/log Green]) are used as extinction coefficients.

Alternatively, the extinction coefficients may be derived without reference to an actual brightfield dye. Instead, a designer may choose a color that should be used for a moderately intense stain. If that color is (R, G, B) in a linear color model wherein the channels R, G, and B are normalized to the interval [0,1], then the extinction coefficients are simply (log R, log G, log B). This approach allows the method to simulate a brightfield stain using a dye that does not exist in nature.

The correspondence of the points in the fluorescent images may then be established by two methods: intensity-based and feature-based.

In a feature-based method, the image of the nuclei, epithelia, stroma or any type of extracellular matrix material may be acquired for both the fluorescent image and the brightfield image. The feature-based structure may be selected using a manual process or automatically. Corresponding structures are selected in images from both modalities. For the fluorescent image, the image may be captured using a fluorescent microscope with an appropriate excitation energy source tuned to a given biomarker and with filters appropriate for collecting the emitted light. A brightfield image of the sample may then be obtained which may then be segmented into Red (R), Green (G) and Blue (B) channels and the color and intensity of the feature-based structure measured.

In an intensity-based method, location of the sample area under the microscope may be controlled with electronic, magnetic, optical or mechanical sensors so that the sample area can be repeatedly located close to the same position for the next image acquisition. Intensity based registration is generally applicable to a broad class of biomarkers. Generally, the biological sample, which is fixed or otherwise provided on a substrate such as, but not limited to, a TMA, a slide, a well, or a grid, is labeled with molecular biomarkers, and imaged through a fluorescent microscope.

In either the intensity-based or feature-based method, the transformation from the fluorescent images to the brightfield color space uses the estimated mapping parameter in a nonlinear transformation equation. The nonlinear transformation equation may be represented using the red, green, blue values or color space (R, G, B) and the transformation represented by the formulas:

R=255exp(−a[Dye1]*z[Dye1]−a[Dye2]z[Dye2]− . . . )

G=255exp(−b[Dye1]*z[Dye1]−b[Dye2]z[Dye2]− . . . )

B=255exp(−c[Dye1]*z[Dye1]−c[Dye2]z[Dye2]− . . . )

In the formulas, the scalars z[Dye1], z[Dye2], . . . are the fluorescent dye quantities observed at a given pixel location. The triples (a[Dyen], b[Dyen], c[Dyen]) are a constant times the extinction coefficients of the nth dye in the virtual stain as defined using a preselected or desired color. The constant is chosen so that the output color values (R, G, B) display a readable range of contrast in the image. R, G, and B are resulting red, green and blue pixel values in the brightfield type image; z is a scaling coefficient for fluorescent dye quantities observed at a given pixel location; and a, b, and c are the extinction coefficients corresponding to the brightfield color space. and wherein the triples a[Dyen], b[Dyen], c[Dyen], are a constant times the extinction coefficients of the nth dye in the virtual stain as defined using a preselected or desired color.

Preferably, the 0.995 quantiles are found for z[Dye1], z[Dye2], . . . , and the constants are chosen such that:

min(exp(−a[Dyen]*z[Dyen]),exp(−b[Dyen]*z[Dyen]),exp(−c[Dyen]*z[Dyen]))=1/255.

This causes the dynamic range of the output color to nearly fill the possible dynamic range of an 8-bit image, and results in an intense contrast.

A sharpening transform may be applied to the virtual stain image after it is synthesized. In one embodiment, the sharpening transform may be implemented as a linear convolution filter whose kernel is the matrix:

$$[ \begin{matrix}- 0.25 & - 0.25 & - 0.25 \\ - 0.25 & 3.00 & - 0.25 \\ - 0.25 & - 0.25 & - 0.25\end{matrix} ]$$

Applying the sharpening transform gives the output image a crisper appearance with sharper edges and more visible fine details.

Once the transformation parameters are calculated, one or more selected areas of the sample may be used for transformation from a set of fluorescent images into a VSI using the virtual H&E mapping or a similar visual image such as brown DAB staining. The molecular biomarkers advantageously provide functional and compartmental information that is not visible using a brightfield image alone. For example, image analysis algorithms can benefit from the added channels to separate the sample compartments while still providing a pathologist or operator image intensity values representative of a brightfield modality (H&E). For example, a VSI representative of a DAB staining protocol for keratin would show cell nuclei in shades of purple and the cytoskeleton of epithelial cells and fibroblasts in shades of brown.

Alternatively, once the mapping parameters are estimated, the transformation algorithm may be applied to other fluorescent images to generate a VSI. The other fluorescent images may be from a different area of the same biological sample. Alternatively, the other fluorescent images may be from a different biological sample. The different biological sample may include a collection of similar cells obtained from tissues of biological subjects that may have a similar function.

Thus, the method for generating a brightfield type image comprises the steps of acquiring image data of two or more fluorescent images of a fixed area on a biological sample, analyzing the image data utilizing, at least in part, feature-based information or pixel intensity data information to generate mapping parameters wherein the mapping parameters comprises a nonlinear estimation model, applying the mapping parameters to the fluorescent images, transforming the two or more fluorescent imaging into a brightfield color space and generating a brightfield type image. The method may further include applying a sharpening transformation correction to the brightfield type image.

### EXAMPLES

The following examples are intended only to illustrate methods and embodiments in accordance with the invention, and as such should not be construed as imposing limitations upon the claims.

### Example 1

In one implementation of this invention, lymph node biopsy from patient suspected of having classical Hodgkin lymphoma is obtained from lymph node excision and examined by standard histology methods: the tissue sample is fixed in 10% neutral buffered formalin for 8 hours, and then dehydrated by passage of series of solutions with increasing ethanol concentration (50%, 75%, 80%, 95%, 100%) followed by xylene. The sample is then embedded in paraffin and sections of four micrometer thickness are sectioned using a microtome. Sections are floated onto a waterbath and collected one at a time onto a standard microscope slide. The slides are allowed to dry and baked for 2 hours in a 60° C. oven and then deparaffinized by passage through xylene, then re-hydrated by passage through ethanol followed by a series of water-ethanol mixtures with decreasing ethanol concentration, and finally washed with PBS.

Next, the slide is subjected to antigen retrieval procedure by heating the slide in Bond Epitope Retrieval solution (Leica) at 100° C. for 20 min. Slide is then stained with CD30 antibody conjugated with Cy5 combined with CD45 antibody conjugated with Cy3, followed by counterstaining with DAPI. The slide is coverslipped and entire slide area is imaged using fluorescence microscope equipped with 1.25× magnification objective and a DAPI filterset. The images are captured using a digital monochrome camera, and then computationally combined to form one stitched image of the entire slide. From this stitched full slide image, location of the tissue section is determined, and coordinates for the tissue section only are recorded. This method significantly shortens the time necessary to collect subsequent images.

10× Images of the tissue section area are then collected using DAPI, Cy3 and Cy5 filtersets to get images specific for nuclei, CD45 and CD30 protein staining, respectively. These individual marker images, after stitching, are overlaid to form a fluorescence pseudocolor image as well as virtual H&E and virtual DAB images. Stitched, combined images allow a computer program or a pathologist to select 5-20 regions of interest from the tissue section that contain tumor cells (e.g., CD30 positive regions). Coordinates for these tumor cell regions are recorded and used to collect images using 40× magnification and filtersets for all fluorophores, including DAPI, as before.

Slide is then subjected to dye inactivation procedure as described more fully in U.S. patent application Ser. No. 13/336,409 entitled “PHOTOACTIVATED CHEMICAL BLEACHING OF DYES FOR USE IN SEQUENTIAL ANALYSIS OF BIOLOGICAL SAMPLES” and filed on Dec. 23, 2011, herein incorporated by reference in its entirety, and stained with antibodies for CD20 and CD15 conjugated with Cy3 and Cy5, respectively and was counterstained with DAPI. Tissue section is aligned so that images would be collected on same regions of interest as on the previous round. Next, the regions of tissue section that are CD30 positive (ROIs) are imaged using coordinates recorded in the first imaging step. Image sets are recorded at 40× using filtersets specific for Cy3, Cy5 and DAPI.

Immunofluorescence image sets are then aligned using DAPI images from each round, respectively, and then overlaid and visualized using specialized visualization software. This allows simultaneous visualization of cell nucleus as well as expression of CD30, CD45, CD20 and CD15.

Subsequently, the slide is subjected to dye inactivation procedure and the process is reiterated for other markers of interest, such as CD3, Pax-5, CD79A, BOB1 or OCT-2. Depending on the dyes used, one, two or several markers can be analyzed at the same time in a single iteration of the process.

This method allows precise identification of the tumor area and cell to cell comparison of expression of the biomarkers tested.

### Example 2

### Multiplexed Analysis of Tissue Samples and

### Diagnosis of Classic Hodgkin Lymphoma

### Methods:

### Specimens

Tissue microarray slides were run in triplicates. The TMA, noted as AGTA744, consisted of 28 cores: (12) non-Hodgkin cores, (12) Hodgkin Lymphoma cases, (2) tonsils, and (2) reactive lymphoma cases. After initial proof of principle study using the tissue microarrays, a total of (8) whole mount formalin fixed paraffin embedded tissue slides was employed in this study. Of the 8 samples, 4 were from biopsies previously diagnosed as classical Hodgkin lymphoma based on standard brightfield immunohistochemistry analyses. Three of the remaining 4 samples were non-Hodgkin lymphomas with CD30+ cells, while one case had been diagnosed as lymphocyte predominance Hodgkin lymphoma, in which CD30+ cells were present. All specimens were cut at 4 uM onto standard glass slides.

TABLE 1
Tissue Microarray Maps
AGTA 744

| marker | | 1 | 2 | 3 | 4 |
|---|---|---|---|---|---|
| placenta | 1 | AGI-4045 | AGI-4046 | AGI-4049 | AGI-4061 |
| | | NHL | NHL | HL | NHL |
| | 2 | AGI-4067 | AGI-4066 | AGI-4065 | AGI-4064 |
| | | NHL | HL | HL | HL |
| | 3 | AGI-4068 | AGI-4069 | AGI-4070 | AGI-4053 |
| | | NHL | HL | HL | HL |
| | 4 | AGI-4055 | AGI-4057 | AGI-4058 | AGI-4051 |
| | | HL | NHL | NHL | HL |
| | 5 | 89-MCH- | 89-HH- | 89-HH- | 89-HH- |
| | | 300-1 | 6271-1B | 3319-A7 | 5092-1B |
| | | NHL | HL | TONSIL | TONSIL |
| | 6 | 92-MC- | 94-HH- | 95-HH- | 90-HH- |
| | | 878-3 | 11015-1B | 4894-A3 | 6842-1A |
| | | HL | NHL | NHL | Lymphoma NOS |
| | 7 | 92-MC- | 94-HH- | 95-HH- | 90-HH- |
| | | 878-3 | 11015-1B | 4894-A3 | 6842-1A |
| | | HL | NHL | NHL | Lymphoma NOS |

NHL: non-Hodgkin lymphoma; HL: Hodgkin lymphoma; TONSIL: control tissue; Lymphoma NOS: lymphoma the diagnosis of which could not be sub-classified, NOS = Not Otherwise Specified.

### Slide Processing & Imaging:

The slides were baked at 60° C. for 1 hour and then deparaffinized and rehydrated through a series of xylene and alcohol washes. The slides were subjected to a two-step, citrate pH6.0 and Tris pH 8.5 antigen retrieval method via standard pressure cooker methods. The slides were blocked with a generic protein solution and stained with DAPI. Afterwards the slides were mounted with a glycerol based mounting media, coverslipped, and the background autofluorescence was captured by imaging on the InCell 2000 fluorescent microscope. For the background imaging, images were acquired in all (4) channels using a 10× objective at the following exposure times: DAPI (250 ms), FITC (1000 ms), cy3 (500 ms), and cy5 (5000 ms).

Following the background imaging, the coverslips were removed by gentle agitation in a PBS bath. Next, the first round, antibody staining for CD30 occurred on the Leica Bond Max, this antibody incubation and all subsequent incubations occurred for 1 hour at room temperature. The concentrations of antibodies employed for each round is highlighted in the table shown in table 2 and the multiplexing order of the antibodies is detailed in table 3. Following the CD30 staining the slides were remounted, coverslipped, and placed back on the InCell 2000 for acquisition of the 10× whole slide image. Using the 10× CD30 image as a guide, the pathologist selected 5-20 regions of interest for further higher magnification, 40× imaging. Once these regions of interest were selected, these formed the x,y,z coordinates on the microscope for the downstream imaging of CD30 and all other targets employed in the multiplexing. Following the 40× imaging of the CD30, the slides were decoverslipped and the fluorescence was deactivated with a solution of NaHCO₃ and H₂O₂for 15 minutes. The slides were re-imaged at the 40× regions of interest after the deactivation process to not only ensure full inactivation of the fluorescence but also these images served as background images for processing of subsequent rounds of staining. Post imaging of the inactivated stain, the slides were placed back on the Leica Bond for the staining of the CD15 antibody. This sequence of staining, imaging, inactivation, and imaging was carried out for a total of 6 rounds as detailed in table 3. DAPI was present in all imaging steps and we also acquired DAPI and FITC channels in all imaging rounds.

TABLE 2
Antibodies & Staining Concentrations Employed

| | | | Staining |
|---|---|---|---|
| | | | Concentration |
| Target | Vendor | Catalog # | (ug/mL) |
| BOB1 | Santa Cruz | sc-955 | 10 |
| CD15 | Dako | AO952 | 1 |
| CD20 | Epitomics | 1632-X | 5 |
| CD3 (1) | Dako | M7254 | 5 |
| CD30 | Leica | NCL-L-CD30 | 6.5 |
| CD45 | Dako | M0701 | 10 |
| CD79 | Dako | M7050 | 8 |
| Oct-2 (C-20) | Santa Cruz | sc-233 | 10 |
| Pax-5 | Epitomics | AC-0158 | 10 |
| Mouse IgG Cy3 | Jackson Immuno | 715-165-150 | 15 |
| Mouse IgM Cy3 | Jackson Immuno | 115-165-075 | 15 |

The Dako CD3 antibody recognizes CD30epsilon as the antigen.

TABLE 3
Multiplexing Order of Antibody Staining

| Round | Antibody Stained |
|---|---|
| Rd1= | CD30 Primary/Secondary |
| Rd2= | CD15 Primary/Secondary |
| Rd3= | CD20 DC Cy3 + Pax-5 DC Cy5 |
| Rd4= | CD45 DC Cy3 + CD3 DC Cy5 |
| Rd5= | CD79a DC Cy3 + Oct2 DC Cy5 |
| Rd6= | BOB1 DC Cy5 only |

### Image Processing & Generation:

Post acquisition, images were taken through a number of algorithms. The algorithms were used to subtract out background autofluorescence, register the images, normalize the images and produce the virtual H&E and virtual DAB blends. The pathologist was able to view each biomarker as a standard grayscale (monochromatic image), an overlay of 2 or more biomarkers, a virtually created virtual H&E, or as a virtually created virtual diaminobenzidine (DAB) image. See U.S. Pat. No. 8,269,827 and U.S. patent application Ser. No. 13/211,725.

### Results and Discussion:

### Proof of Principle Using Tissue Microarray AGTA 744:

From a paraffin block, thirteen sections were sectioned, each of four micrometer thickness, using a microtome. Three of the sections were subjected to multiplexed staining with all 9 antibodies. Nine slides were only stained by a single antibody. The slides were kept at 4° C. until their appropriate round of staining. At each round of staining, the corresponding singleplexed slide was decoverslipped and stained alongside the multiplex round.

Thus, the three multiplexed slides were first subjected to staining with CD30 primary antibody and Cy3 conjugated secondary antibody, followed by counterstaining with DAPI. One of the nine singleplexed slides was subjected to staining together with the three multiplexed slides.

Thus, for the second round, the three multiplexed slides were subjected to staining with CD15 primary antibody and Cy3 conjugated secondary antibody, followed by counterstaining with DAPI. The second of the nine singleplexed slides was subjected to staining together with the three multiplexed slides.

Thus, for the third round, the three multiplexed slides were subjected to staining with CD20 antibody conjugated with Cy3 combined with Pax-5 antibody conjugated with Cy5, followed by counterstaining with DAPI. The third and fourth of the nine singleplexed slides were subjected to staining together with the three multiplexed slides. However, in addition to DAPI staining, the third slide was only stained with CD20 antibody conjugated with Cy3; while the fourth slide was only stained with Pax-5 antibody conjugated with Cy5.

For round four, five and six, the multiplexed and singleplexed slides were similarly processed, with CD45 antibody conjugated with Cy3 and CD3 antibody conjugated with Cy5 (round four), CD79A Cy3 and Oct2 Cy5 (round five), while BOB1 Cy5 was used for the last round.

We obtained comparable results in both the staining specificity and intensity for the multiplexed and singleplexed slides, for all the nine antibodies used. Thus, our multiplexed method is capable of detecting all of the nine biomarkers on a single slide section.

Results from Whole Mount Tissue Slides:

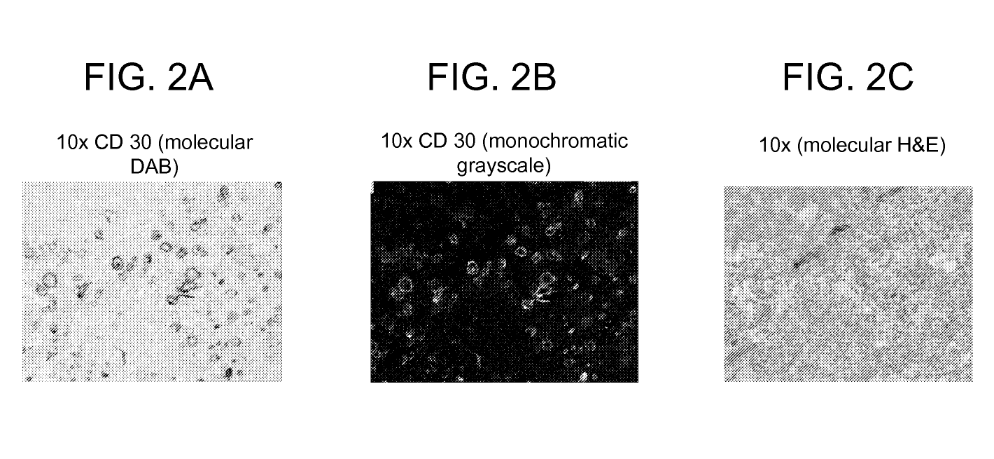

FIG. 2 shows representative 10× images from a Hodgkin lymphoma sample illustrating the various visualization techniques presented to the pathologist. All images presented are from the same field of view in the tissue sample. A: CD 30 staining presented as a virtual DAB image. The brown (darker) staining is the CD30 staining and the blue (lighter) color represent the nuclei (psuedo hematoxylin staining from DAPI staining). B: CD 30 staining presented as a monochromatic, grayscale image. The gray color represents the CD30 positive areas. C. A virtual H&E image which shows the overall morphology of the tissue.

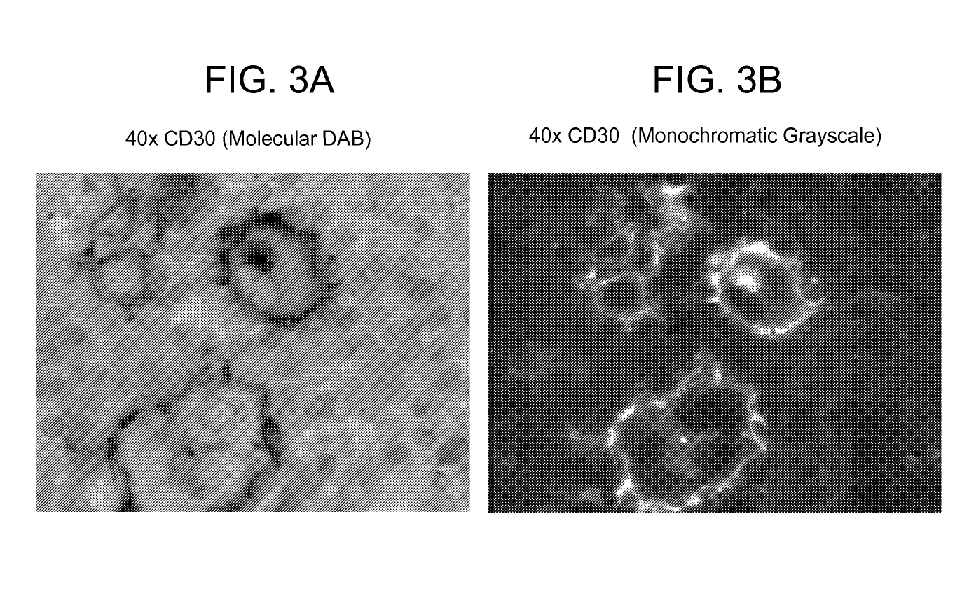

FIG. 3 shows representative 40× images from a Hodgkin lymphoma sample comparing the two fundamental ways that a single biomarker can be presented to a pathologist. Both images presented are from the same field of view in the tissue sample. A: CD 30 staining presented as a virtual DAB image. The brown (darker) staining is the CD30 staining and the blue (lighter) color represent the nuclei (psuedo hematoxylin staining from DAPI staining). B: CD 30 staining presented as a monochromatic, grayscale image. The gray color represents the CD30 positive areas.

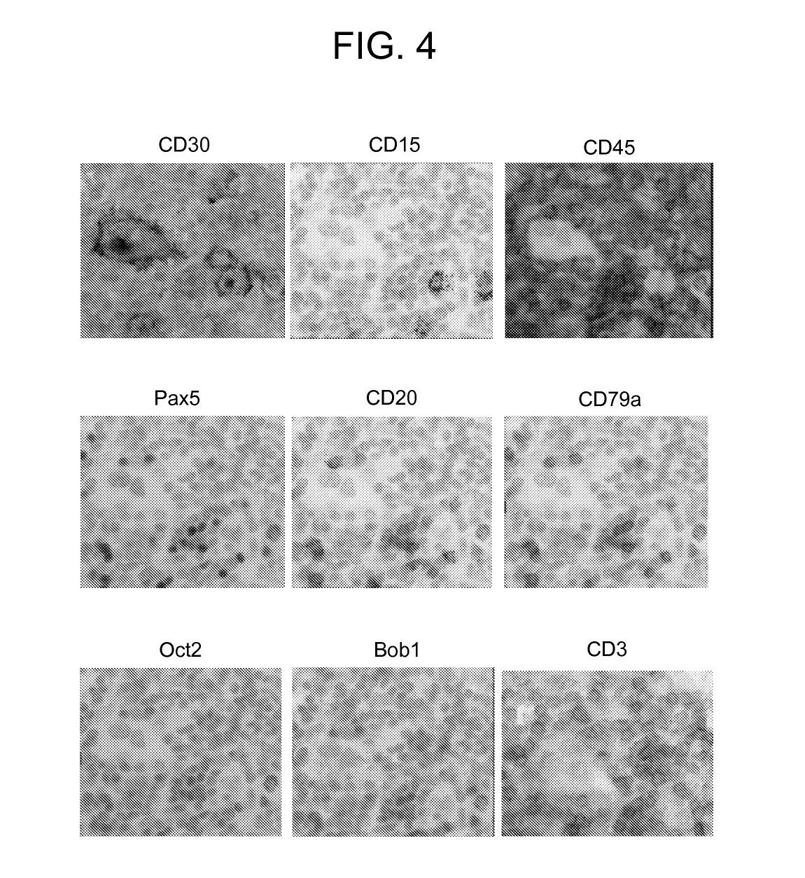

FIG. 4 shows representative 40× virtual DAB images from a Hodgkin lymphoma sample illustrating the results of multiplexing all nine antibodies on a single tissue section. The nine antibodies evaluated were: CD30, CD15, CD45, Pax5, CD20, CD79a, Oct2, BOB1, and CD3. The brown (darker) color represents areas that are positive for that particular biomarker and the blue (lighter) color represents the nuclei (pseudo hematoxylin from DAPI staining) staining. Images presented are all from the same field of view in the tissue sample

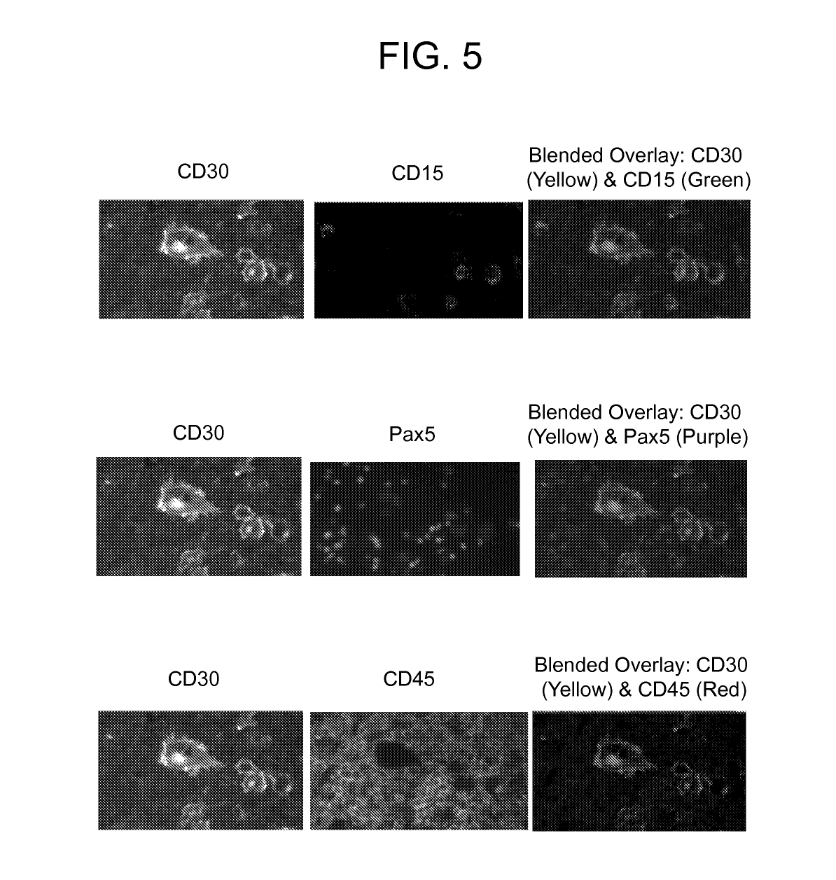

FIG. 5 shows representative 40× images from a Hodgkin lymphoma. Row1: CD30 and CD15 shown as a monochromatic grayscale image then as a blended overlay of both channels. In the blended overlay, CD30 is represented in yellow and CD15 is green. Row2: CD30 and Pax5 shown as a monochromatic grayscale image then as a blended overlay of both channels. In the blended overlay, CD30 is represented in yellow and Pax5 in purple. Row3: CD30 and CD45 shown as a monochromatic grayscale image then as a blended overlay of both channels. In the blended overlay, CD30 is represented in yellow and CD45 is red. All images are from the same field of view in the tissue.

A correct diagnosis of classical Hodgkin lymphoma vs. other condition was able to be made using the novel multiplex immunofluorescent platform in all cases. Subjectively, the pathologist noted that the novel methodology allowed for a significantly more confident assessment of marker expression on the Hodgkin cells in the four cases of classical Hodgkin lymphoma, eliminating many issues of staining ambiguity and allowing recognition of subtle (weak vs. negative) nuances of staining intensity in the cells of interest. The CD30+ cells in the 4 other cases clearly showed a B cell profile using the novel platform that was easily distinguishable from the classical Hodgkin cell phenotype

Despite there being traditional methods for the diagnosis of Hodgkin lymphoma, there are limitations to these methods. This new method of fluorescent multiplexing on a single tissue section allows more accurate interpretation of the biomarker expression profile of all antibodies of interest on the same Hodgkin cell, obviating many ambiguities in stain interpretation. It is likely that this paradigm can be expanded to a greater range of challenging cases in Hematopathology.

### Example 3

### Multiplexed Analysis of Tissue Samples and Benchmark to the Traditional IHC Methodology

### Methods:

### Specimens

A total of 40 whole mount, formalin fixed paraffin embedded tissues were employed to evaluate antibody specificity of the fluorescent stains and benchmark this staining to the traditional IHC methodology. Based on historical characterization from traditional brightfield IHC analyses the tissues were characterized as follows: 19 classical Hodgkin Lymphoma cases, 5 reactive lymph node cases, 7 B-Cell lymphoma cases, 1 nodular lymphocyte predominant lymphoma, 1 plasma cell lymphoma, and 3 T-Cell lymphoma cases, 2 tonsil, and 2 breast carcinomas

### Slide Processing & Imaging:

The slides were baked at 60° C. for 1 hour and then deparaffinized and rehydrated through a series of xylene and alcohol washes. The slides were subjected to a two-step, citrate pH6.0 and Tris pH 8.5 antigen retrieval method via standard pressure cooker methods. The slides were blocked with a generic protein solution. Afterwards the slides were stained with DAPI and mounted with a glycerol based mounting media, coverslipped, and the background autofluorescence was captured by imaging on the InCell 2000 fluorescent microscope. For the background imaging, images were acquired in all (4) channels using a 10× objective at the following exposure times: DAPI (250 ms), FITC (1000 ms), cy3 (500 ms), and cy5 (5000 ms).

Following the background imaging, the coverslips were removed by gentle agitation in a PBS bath. Next, the first round, antibody staining for CD30 and BOB1 occurred on the Leica Bond Max, this antibody incubation and all subsequent incubations occurred for 1 hour at room temperature. The concentrations of antibodies employed for each round is highlighted in the table shown in table 4 and the multiplexing order of the antibodies is detailed in table 5. Following the CD30 and BOB1 staining the slides were remounted, coverslipped, and placed back on the InCell 2000 for acquisition of the 10× whole slide image for CD30. Using the 10× CD30 image as a guide, the pathologist selected 5-20 regions of interest for further higher magnification, 40× imaging. Once these regions of interest were selected, these formed the x,y,z coordinates on the microscope for the downstream imaging of CD30 and all other targets employed in the multiplexing. Following the 40× imaging of the CD30 and BOB1 the slides were decoverslipped and the fluorescence was deactivated with a solution of NaHCO₃ and H₂O₂ for 15 minutes. The slides were re-imaged at the 40× regions of interest after the deactivation process to not only ensure full inactivation of the fluorescence but also these images served as background images for processing of subsequent rounds of staining. Post imaging of the inactivated stain, the slides were placed back on the Leica Bond for the staining of the CD15 and OCT2 antibodies. This sequence of staining, imaging, inactivation, and imaging was carried out for a total of 5 rounds as detailed in table 5. DAPI was present in all imaging steps and we also acquired DAPI and FITC channels in all imaging rounds.

TABLE 4
Antibodies & Staining Concentrations Employed

| | | | Staining |
|---|---|---|---|
| | | | Concentration |
| Target | Vendor | Catalog # | (ug/mL) |
| BOB1 | Santa Cruz | sc-955 | 10 |
| CD15 | Dako | AO952 | 1 |
| CD20 | Epitomics | 1632-X | 5 |
| CD3 (1) | Dako | M7254 | 5 |
| CD30 | Leica | NCL-L-CD30 | 13 |
| CD45 | Dako | M0701 | 10 |
| CD79 | Dako | M7050 | 8 |
| Oct-2 (C-20) | Santa Cruz | sc-233 | 10 |
| Pax-5 | Epitomics | AC-0158 | 10 |
| Mouse IgG Cy3 | Jackson Immuno | 115-166-062 | 10 |
| Mouse IgM Cy3 | Jackson Immuno | 115-165-075 | 10 |

(1) The Dako CD3 antibody recognizes CD30epsilon as the antigen.

TABLE 5
Multiplexing Order of Antibody Staining

| Round | Antibody Stained |
|---|---|
| Rd1= | CD30 Primary/Secondary + Cy5 BOB1 |
| Rd2= | CD15 Primary/Secondary + Cy5-OCT2 |
| Rd3= | CD20 DC Cy3 + Pax-5 DC Cy5 |
| Rd4= | CD45 DC Cy3 + CD3 DC Cy5 |
| Rd5= | CD79a DC Cy3 |

### Image Processing & Generation:

See Example 2 above for detail.

### Results and Discussion:

Images obtained in this example are comparable to those of Example 2 above. Table 6 shows the staining specificity concordance between the multiplexed immunofluorescence method and the traditional brightfield IHC method for each of the biomarkers. A pathologist reviewed either the virtual DAB image created from the fluorescent antibody stain or a traditional DAB IHC image from a serial tissue section on a total of 40 whole mount formalin fixed tissues. Listed in each row in Table 6 are the concordance levels for each of the biomarkers. The fraction in parentheses highlights the number of cases that were evaluated by the pathologist; in the instances where there were less than 40 cases evaluated or where there was less than 100% concordance, notes have been included below the table to explain.

TABLE 6
Concordance of Immunofluorescence Stains Compared
to Traditional Brightfield IHC for each Biomarker

| Biomarker | Concordance | |
|---|---|---|
| CD30 | 100% | (40/40) |
| CD15 | 97.4% | (38/39) ^{1, 2} |
| CD20 | 100% | (40/40) |
| Pax5 | 100% | (40/40) |
| CD79a | 100% | (39/39)³ |
| Oct2 | 100% | (38/38) ^{2, 4, 5} |
| BOB1 | 97.4% | (38/39) ^{4, 6} |
| CD45 | 100% | (39/39)³ |
| CD3 | 100% | (39/39)³ |

¹ Case #20862-1 exhibited non-specific blotches of staining for CD15 in IF which are believed to be non-specific.
² IF Images for the CD15 and Oct2 imaging round were out of focus for case #16409 and were therefore not scored.
³The IF slide for case #20862-1 was damaged beyond repair prior to staining CD45, CD3, and CD79a and was therefore not scored for those biomarkers.
⁴ No relevant tissue is visible on the IHC images of BOB1 and Oct2 for case #24750, the images were not scored.
⁵ Both the IF and the IHC were deemed to be non-specific for case #17993.
⁶ Case #349 exhibited BOB1 staining in a different cell population than is expected and was believed to be non-specific.

### Example 4

### Multiplexed Analysis of Tissue Samples for the

Diagnosis of Classic Hodgkin Lymphoma

Methods:

### Specimens

A total of 30 whole mount, formalin fixed paraffin embedded tissues were tested to evaluate the pathologist's ability to render a diagnosis of Hodgkin lymphoma versus other conditions. Based on historical characterization from traditional brightfield IHC analyses the tissues were characterized as follows: 4 cases of Hodgkin lymphoma run in replicate to yield 11 total slides, 4 cases of B-cell lymphoma run in replicate to yield 13 total slides, and 3 cases of T-cell lymphoma run in replicate to yield 6 slides.

### Slide Processing & Imaging:

See Example 2 above for details.

### Image Processing & Generation:

See Example 2 above for details.

### Results and Discussion:

Images obtained in this example are comparable to those of Example 2 above. Table 7 highlights the concordance in final diagnoses between the multiplexed analyses and the traditional brightfield IHC method on a case level. A correct diagnosis of classical Hodgkin lymphoma vs. other condition was able to be made using the novel multiplex immunofluorescent platform in all cases. Table 7 also highlights the concordance on a slide level when multiple replicates of the same case were evaluated with the multiplexed analysis. There was 100% concordance at the slide and patient levels for T-cell and Hodgkin's Lymphoma diagnoses. The concordance of B-cell Lymphoma diagnoses was 62% at the slide level and 75% at the patient level. Of the four diagnoses of B-cell Lymphoma by Traditional Brightfield IHC, one patient was believed to be misdiagnosed due to the limitations of the Brightfield method.

TABLE 7
Diagnostic Concordance Levels between Multiplexed
Analysis and Traditional Brightfield IHC (Case
Level) or Concordance Levels of Replicates Evaluated
with the Multiplexed Analysis (Slide Level)

| Diagnosis | Case Level | Slide Level |
|---|---|---|
| Hodgkin Lymphoma | 100% | 100% |
| B-Cell Lymphoma | 75% | 62% |
| T-Cell Lymphoma | 100% | 100% |

While the particular embodiment of the present invention has been shown and described, it will be obvious to those skilled in the art that changes and modifications may be made without departing from the teachings of the invention. The matter set forth in the foregoing description and accompanying drawings is offered by way of illustration only and not as a limitation. The actual scope of the invention is intended to be defined in the following claims when viewed in their proper perspective based on the prior art.

What is claimed is:

1. A method of analyzing a biological sample suspected of having classic Hodgkin lymphoma, comprising:
(A) detecting, in a single sample, for the expression of at least two biomarkers selected from CD30, CD15, CD20, CD45, CD3, Pax-5, CD79A, BOB1 and OCT-2; and
(B) analyzing the sample based on the presence, absence and/or expression level of the at least two biomarkers.

2. The method of claim 1, wherein the expression of at least three of said biomarkers is analyzed.

3. (canceled)

4. The method of claim 1, wherein the expression of at least CD30, CD15, CD45, CD3 and Pax-5 is analyzed.

5. The method of claim 1, wherein the expression of all the nine biomarkers is analyzed.

6. (canceled)

7. The method of claim 1, wherein the detecting step also comprises detecting for expression of MUM1, kappa/lambda or pan T-cell markers.

8. The method of claim 1, wherein the expression of the biomarkers are analyzed within individual cells.

9. (canceled)

10. The method of claim 1, wherein the detection of each biomarker comprises contacting the sample with a binder that binds to said biomarker.

11. (canceled)

12. The method according to claim 1, wherein the detecting step comprises:
(1) generating a first series of images of the biological sample, which step comprises:
(a) contacting the sample on a solid support with a first binder for a first of said biomarkers;
(b) staining the sample with a fluorescent marker that provides morphological information;
(c) detecting, by fluorescence, for the presence of signals from the first binder and the fluorescent marker; and
(d) generating the first images of at least part of the sample from the detected fluorescent signals; and
(2) after signal removal from the first binder, generating one or more second series of images of the biological sample, which step comprises:
(a) contacting the same sample with a binder for another of said biomarkers;
(b) optionally staining the sample with a fluorescent marker that provides morphological information;
(c) detecting, by fluorescence, for the presence of signals from the binder and the fluorescent marker; and
(d) generating the second images of at least part of the sample from the detected fluorescent signals.

13. The method according to claim 12, wherein step (1)(d) comprises:
(i) generating initial images of at least part of the sample from the detected fluorescent signals; and
(ii) selecting a region of interest (ROI) from the initial images, and detecting by fluorescence, signals from at least the first binder and the fluorescent marker to generate the first images at a higher resolution than the initial images.

14. The method according to claim 13, wherein step (2)(d) comprises:
(i) obtaining the ROI information from step (1); and
(ii) detecting by fluorescence, signals from at least the binder and the fluorescent marker to generate the second series of images at the same higher resolution as in step (1) above.

15. The method according to claim 12, wherein the first series of images include at least an image from the fluorescent signals from the first binder, an image from the fluorescent marker, and optionally an image that includes the fluorescent signals from the first binder and the fluorescent marker.

16. The method of claim 12, wherein the contacting step (1) (a) includes contacting the sample with a second binder for a second of said biomarkers, and the second binder carries a fluorescent signal separately detectable from the other fluorescent signals in step (1); the first images include an image generated from the fluorescent signals from the second binder and optionally a composite image comprising (i) an image generated from signals from the second binder and the fluorescent marker and/or (ii) the first and second binder and the fluorescent marker.

17. The method of claim 12, wherein the second series of images include at least an image from the fluorescent signals from the binder, an image from the fluorescent marker, and optionally an image that includes the fluorescent signals from the binder and the fluorescent marker.

18. The method of claim 12, wherein the contacting step (2) (a) includes contacting the sample with one or more binder(s) for one or more further of said biomarkers not detected in step (1) and elsewhere in step (2), wherein the further binder(s) each carry a fluorescent signal separately detectable from the other fluorescent signals including from the other binder(s) used in the same step (2).

19. The method according to claim 18, wherein the second images include respective images generated from the fluorescent signals from each further binder(s) and optionally one or more composite images comprising (i) respective images generated from signal from the each further binder(s) and the fluorescent marker; or (ii) an image generated from the signals from each binder in step (2) and the fluorescent marker.

20. The method of claim 12, wherein step (2) is repeated for additional biomarkers until all biomarkers of interest are analyzed, and wherein each step (2) takes place after signal removal from the binders present from the previous step (2).

21. The method of claim 12, wherein the detecting step in generating the first series of images or the detecting step in generating the second series of images further comprises detecting autofluorescense of the biological sample.

22. The method of claim 12, wherein the step of generating the first series of images further comprises: prior to generating the images of the sample, generating a lower resolution image of the entire solid support and locating the sample on the solid support.

23. The method of claim 12, wherein generating the first images and/or generating the second images comprises generating brightfield type images that resembles a brightfield stain.

24. (canceled)

25. (canceled)

26. The method of claim 12, further comprising an antigen retrieval step prior to the contacting step (1)(a).

27. The method of claim 12, wherein said binders are antibodies specific for the target proteins.

28. (canceled)

29. The method of claim 12, wherein said fluorescent marker is selected from the group consisting 4′,6-diamidino-2-phenylindole (DAPI), Eosin, Hoechst 33258 and Hoechst 33342 (two bisbenzimides), Propidium Iodide, Quinacrine, Fluorescein-phalloidin, Chromomycin A 3, Acriflavine-Feulgen reaction, Auramine O-Feulgen reaction or Ethidium Bromide.

30-33. (canceled)

34. The method of claim 12, wherein the first biomarker is CD30, and the ROI selection is guided by signals from the binder for CD30.

35. (canceled)

36. The method of claim 12, wherein the analyzing step also includes an assessment of the morphology of the sample.

37. A method for diagnosis of a classic Hodgkin lymphoma, comprising
(A) detecting, in a single sample, the presence and expression level of CD30, CD15, CD20, CD45, CD3, Pax-5, CD79A, BOB1 and OCT-2;
(B) analyzing the presence and relative expression level of the biomarkers; and
(C) diagnosing whether the patient has classic Hodgkin lymphoma;
wherein the detecting step comprises:
(1) generating a first series of images of the biological sample, which step comprises:
(a) contacting the sample on a solid support with an antibody for CD30;
(b) staining the sample with DAPI;
(c) detecting, by fluorescence, signals from a label of the antibody for CD30 and DAPI; and
(d) generating the first images of at least part of the sample from the detected fluorescent signals;
(2) after signal removal, generating second series of images of the biological sample, which step comprises:
(a) contacting the same sample with an antibody for CD15;
(b) optionally staining the sample with DAPI;
(c) detecting, by fluorescence, signals from a label of the antibody for CD 15 and DAPI; and
(d) generating the second images of at least part of the sample from the detected fluorescent signals; and
(3) repeat step (2) for at least four more rounds, with differentially labeled antibody specific for CD20 and Pax-5, CD45 and CD3, CD79a and Oct2, and labeled antibody for Bob1, respectively.

38. The method according to claim 1, wherein the detecting step comprises:
(1) generating a first image of the biological sample, which step comprises:
(a) contacting the sample on a solid support with a first binder for CD30;
(b) detecting, by fluorescence, for the presence of signals from the first binder; and
(c) generating the first image of at least part of the sample from the detected fluorescent signals; and
(2) after signal removal from the first binder, generating a second image of the biological sample, which step comprises:
(a) contacting the same sample with a binder for another biomarker;
(b) detecting, by fluorescence, for the presence of signals from the binder; and
(c) generating the second image of at least part of the sample from the detected fluorescent signals.

39. The method according to claim 38, wherein step (1)(c) comprises,
(i) generating an initial image of at least part of the sample from the detected fluorescent signals; and
(ii) selecting a region of interest (ROI) from the initial image, and detecting by fluorescence, signals from the first binder to generate the first image at a higher resolution than the initial image.

40. The method according to claim 39, wherein step (2)(c) comprises,
(i) obtaining the ROI information from step (1); and
(ii) detecting by fluorescence, signals from the binder for said another biomarker to generate the second image at the same higher resolution as in step (1) above.

41. The method of claim 38, wherein step (2) is repeated for additional biomarkers until all biomarkers of interest are analyzed, and wherein each step (2) takes place after signal removal from the binders present from the previous step (2).

42-47. (canceled)

48. A method of treatment for a patient having classical Hodgkin lymphoma, comprising diagnosing the patient as having classical Hodgkin lymphoma according to claim 37, and treating the patient with a drug for classical Hodgkin lymphoma.

49. The method of treatment according to claim 48, wherein said drug targets CD30.

50. The method of treatment according to claim 49, wherein said drug is selected from apomab (RG7425), brentuximab vedotin (SGN-35), DCDT2980S, PF-05230905 and tigatuzumab (CS-1008).

51. A method for diagnosis of a classic Hodgkin lymphoma, comprising
(A) detecting, in a single sample, the presence and expression level of CD30, CD15, CD20, CD45, CD3, Pax-5, CD79A, BOB1 and OCT-2;
(B) analyzing the presence and relative expression level of the biomarkers; and
(C) diagnosing whether the patient has classic Hodgkin lymphoma;
wherein the detecting step comprises:
(1) generating a first series of images of the biological sample, which step comprises:
(a) contacting the sample on a solid support with an antibody for CD30 and labeled antibody specific for BOB1;
(b) staining the sample with DAPI;
(c) detecting, by fluorescence, signals from a label of the antibody for CD30, the labeled antibody specific for BOB1 and DAPI, wherein the signals are distinguishable from each other; and
(d) generating the first images of at least part of the sample from the detected fluorescent signals;
(2) after signal removal, generating second series of images of the biological sample, which step comprises:
(a) contacting the same sample with an antibody for CD15 and labeled antibody specific for Oct2;
(b) optionally staining the sample with DAPI;
(c) detecting, by fluorescence, signals from a label of the antibody for CD15, the labeled antibody specific for Oct2 and DAPI, wherein the signals are distinguishable from each other; and
(d) generating the second images of at least part of the sample from the detected fluorescent signals; and
(3) repeat step (2) for at least three more rounds, with differentially labeled antibody specific for CD20 and Pax-5, CD45 and CD3, and labeled antibody for CD79a, respectively.


SEQUENCE LISTING (12 sequences)

SEQ ID NO: 1 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSRARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 2 - protein, 530 aa, Homo sapiens
MRRLWGAARKPSGAGWEKEWAEAPQEAPGAWSGRLGPGRSGRKGRAVPGWASWPAHLALAARPARHLGGAGQGPRPLHSGTAPFHSRASGERQRRLEPQLQHESRCRSSTPADAWRAEAALPVRAMGAPWGSPTAAAGGRRGWRRGRGLPWTVCVLAAAGLTCTALITYACWGQLPPLPWASPTPSRPVGVLLWWEPFGGRDSAPRPPPDCRLRFNISGCRLLTDRASYGEAQAVLFHHRDLVKGPPDWPPPWGIQAHTAEEVDLRVLDYEEAAAAAEALATSSPRPPGQRWVWMNFESPSHSPGLRSLASNLFNWTLSYRADSDVFVPYGYLYPRSHPGDPPSGLAPPLSRKQGLVAWVVSHWDERQARVRYYHQLSQHVTVDVFGRGGPGQPVPEIGLLHTVARYKFYLAFENSQHLDYITEKLWRNALLAGAVPVVLGPDRANYERFVPRGAFIHVDDFPSASSLASYLLFLDRNPAVYRRYFHWRRSYAVHITSFWDEPWCRVCQAVQRAGDRPKSIRNLASWFER

SEQ ID NO: 3 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 4 - protein, 391 aa, Homo sapiens
MDLEKNYPTPRTSRTGHGGVNQLGGVFVNGRPLPDVVRQRIVELAHQGVRPCDISRQLRVSHGCVSKILGRYYETGSIKPGVIGGSKPKVATPKVVEKIAEYKRQNPTMFAWEIRDRLLAERVCDNDTVPSVSSINRIIRTKVQQPPNQPVPASSHSIVSTGSVTQVSSVSTDSAGSSYSISGILGITSPSADTNKRKRDEGIQESPVPNGHSLPGRDFLRKQMRGDLFTQQQLEVLDRVFERQHYSDIFTTTEPIKPEQTTEYSAMASLAGGLDDMKANLASPTPADIGSSVPGPQSYPIVTGRDLASTTLPGYPPHVPPAGQGSYSAPTLTGMVPGSEFSGSPYSHPQYSSYNDSWRFPNPGLLGSPYYYSAAARGAAPPAAATAYDRH

SEQ ID NO: 5 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 6 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 7 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 8 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 9 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 10 - protein, 479 aa, Homo sapiens
MVHSSMGAPEIRMSKPLEAEKQGLDSPSEHTDTERNGPDTNHQNPQNKTSPFSVSPTGPSTKIKAEDPSGDSAPAAPLPPQPAQPHLPQAQLMLTGSQLAGDIQQLLQLQQLVLVPGHHLQPPAQFLLPQAQQSQPGLLPTPNLFQLPQQTQGALLTSQPRAGLPTQAVTRPTLPDPHLSHPQPPKCLEPPSHPEEPSDLEELEQFARTFKQRRIKLGFTQGDVGLAMGKLYGNDFSQTTISRFEALNLSFKNMCKLKPLLEKWLNDAETMSVDSSLPSPNQLSSPSLGFDGLPGRRRKKRTSIETNVRFALEKSFLANQKPTSEEILLIAEQLHMEKEVIRVWFCNRRQKEKRINPCSAAPMLPSPGKPASYSPHMVTPQGGAGTLPLSQASSSLSTTVTTLSSAVGTLHPSRTAGGGGGGGGAAPPLNSIPSVTPPPPATTNSTNPSPQGSHSAIGLSGLNPSTGPGLWWNPAPYQP

SEQ ID NO: 11 - protein, 256 aa, Homo sapiens
MLWQKPTAPEQAPAPARPYQGVRVKEPVKELLRRKRGHASSGAAPAPTAVVLPHQPLATYTTVGPSCLDMEGSVSAVTEEAALCAGWLSQPTPATLQPLAPWTPYTEYVPHEAVSCPYSADMYVQPVCPSYTVVGPSSVLTYASPPLITNVTTRSSATPAVGPPLEGPEHQAPLTYFPWPQPLSTLPTSTLQYQPPAPALPGPQFVQLPISIPEPVLQDMEDPRRAASSLTIDKLLLEEEDSDAYALNHTLSVEGF

SEQ ID NO: 12 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR